(19) United States
(12) Patent Application Publication
(10) Pub. No.: US 2024/0342450 A1
(43) Pub. Date: Oct. 17, 2024

(54) DEVICE FOR SPRAYING CELLS AND POLYMER AND USES THEREOF

(71) Applicants: Children's National Medical Center, Washington, DC (US); University of Maryland, College Park, College Park, MD (US)

(72) Inventors: Anthony Sandler, Bethesda, MD (US); Reza Monfaredi, Rockville, MD (US); Tyler Salvador, Silver Spring, MD (US); Michele Saruwatari, Washington, DC (US); Peter Kofinas, North Bethesda, MD (US)

(73) Assignees: Children's National Medical Center, Washington, DC (US); University of Maryland, College Park, College Park, MD (US)

(21) Appl. No.: 18/638,604

(22) Filed: Apr. 17, 2024

Related U.S. Application Data
(60) Provisional application No. 63/496,472, filed on Apr. 17, 2023.

(51) Int. Cl. A61M 35/00; A61L 26/00; A61M 11/00

(52) U.S. Cl. CPC A61M 35/003; A61L 26/0052; A61L 26/0057; A61L 26/0076; A61L 26/0095; A61M 11/007

(57) ABSTRACT

A solution blow spin device includes a syringe pump assembly, an airbrush adapter, and a handle base. The syringe pump assembly, airbrush adapter, and handle base are reversibly coupled to one another and configured to work in tandem to address wound dressing using a polymer solution and autologous skin cell suspension co-spray. The airbrush adapter includes multiple nozzles for deposition of multiple solutions sequentially or simultaneously in order to improve would dressing efficacy. The syringe pump assembly includes multiple syringes holding the multiple solutions, and the syringes can be emptied by individually addressable lead screws configured to translate blocks to depress syringe plungers when the lead screws are rotated via corresponding actuators.

### CROSS-REFERENCE TO RELATED APPLICATIONS

The present application claims priority to U.S. Provisional Application No. 63/496,472, filed Apr. 17, 2023, which is incorporated herein by reference in its entirety for all purposes.

### BACKGROUND

### Field of the Disclosure

The present disclosure relates to a solution blow spin device.

### Description of the Related Art

Autologous skin cell suspensions (ASCS) are useful for treating burn wounds, including in adjunct with split thickness skin grafts (STSG) for full thickness injuries. However, when ASCS and dressing application are performed in a stepwise fashion, the ASCS can leak out of the wound bed. A simultaneous application process could theoretically limit ASCS loss, streamline the wound care procedure and improve healing outcomes.

### SUMMARY

The foregoing paragraphs have been provided by way of general introduction and are not intended to limit the scope of the following claims. The described embodiments, together with further advantages, will be best understood by reference to the following detailed description taken in conjunction with the accompanying drawings.

In one embodiment, the present disclosure is related to a solution blow spin device including a parameter input interface configured to receive an input from a user, a first lead screw mechanically coupled to a first actuator and a second lead screw mechanically coupled to a second actuator, the first actuator and the second actuator each being electrically connected to the parameter input interface, the second lead screw being disposed proximal and parallel to the first lead screw, a first syringe coupled to a first syringe holder and a second syringe coupled to a second syringe holder, the first syringe holder and the second syringe holder disposed at a first end of the syringe pump assembly, a plunger of the first syringe extending out from the first syringe and a plunger of the second syringe extending out from the second syringe, the first syringe configured to hold a first solution and the second syringe configured to hold a second solution, the second syringe being disposed proximal and parallel to the second syringe, a first block coupled to the first lead screw, the first block configured to abut the plunger of the first syringe and translate along a direction of the first lead screw upon rotation of the first lead screw, and a second block coupled to the second lead screw, the second block configured to abut the plunger of the second syringe and translate along a direction of the second lead screw upon rotation of the second lead screw; and an airbrush adapter.

In one embodiment, the airbrush adapter includes an adapter base including a first portion, an open portion, and a second portion, the first portion of the adapter base coupling with the first end of the syringe pump assembly, a first syringe fitment configured to receive a tip of the first syringe, the first syringe fitment being an opening extending through the first portion, a first hollow needle fluidly connected to the first syringe, the first hollow needle disposed in a hollow nozzle body, the first hollow needle having a tip arranged through a first opening at a tip of the hollow nozzle body, the tip of the first hollow needle being concentric with the first opening but spaced apart from the first opening, the first hollow needle configured to eject the first solution through the tip of the first hollow needle, a second syringe fitment configured to receive a tip of the second syringe, the second syringe fitment being an opening extending through the first portion, a second hollow needle fluidly connected to the second syringe, the second hollow needle disposed in the hollow nozzle body, the second hollow needle having a tip arranged through a second opening at the tip of the hollow nozzle body, the tip of the second hollow needle being concentric with the second opening but spaced apart from the second opening, the second hollow needle configured to eject the second solution through the tip of the second hollow needle, and an air channel inlet fluidly coupled to the hollow nozzle body, the air channel inlet configured to receive a pressurized gas, the hollow nozzle body configured to eject the pressurized gas through the first opening around the tip of the first hollow needle and the second opening around the tip of the second hollow needle.

In one embodiment, the solution blow spin device further includes a handle base, including a first portion configured to couple with the syringe pump assembly and a second portion configured to fluidly couple with the airbrush adapter, and a handle attached to the first portion of the handle base, the handle including a push button configured to actuate the first actuator and the second actuator when depressed.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1A is a perspective view schematic of a blow spin device, according to an embodiment of the present disclosure.

FIG. 1B is a perspective view schematic of a blow spin device, according to an embodiment of the present disclosure.

FIG. 2 is a top-down view schematic of a blow spin device, according to an embodiment of the present disclosure.

FIG. 3 is a cross-sectional schematic of a blow spin device, according to an embodiment of the present disclosure.

FIG. 4 is an exploded view schematic of a blow spin device, according to an embodiment of the present disclosure.

FIG. 5 is a perspective view schematic of a multi-syringe pump, according to an embodiment of the present disclosure.

FIG. 6 is a perspective view schematic of a multi-syringe pump, according to an embodiment of the present disclosure.

FIG. 7 is an exploded view schematic of a multi-syringe pump, according to an embodiment of the present disclosure.

FIG. 8 is a perspective view schematic of a multi-nozzle airbrush adapter, according to an embodiment of the present disclosure.

FIG. 9 is a top-down and cross-sectional view schematic of a multi-nozzle airbrush adapter, according to an embodiment of the present disclosure.

FIG. 10 is an exploded view schematic of a multi-nozzle airbrush adapter, according to an embodiment of the present disclosure.

FIG. 11 is a top-down and cross-sectional view schematic of a multi-nozzle airbrush adapter body, according to an embodiment of the present disclosure.

FIG. 12 is an exploded view schematic of a multi-nozzle airbrush adapter body, according to an embodiment of the present disclosure.

FIG. 13 is a perspective view schematic of a base with handle, according to an embodiment of the present disclosure.

FIG. 14 is a perspective view schematic of a base with handle, according to an embodiment of the present disclosure.

FIG. 15 is images of mice wound dressing, according to an embodiment of the present disclosure.

FIG. 16 is a plot describing cell viability as a function of spray pressure, according to an embodiment of the present disclosure.

FIG. 17 is a plot describing epithelial tongue length for various dressings, according to an embodiment of the present disclosure.

FIG. 18 is a schematic view of user devices communicatively connected to a server, according to an embodiment of the present disclosure.

FIG. 19 is a block diagram illustrating an electronic user device, according to an embodiment of the present disclosure.

FIG. 20 is a schematic of a hardware system for performing a method, according to an embodiment of the present disclosure.

FIG. 21 is a schematic of a hardware configuration of a device for performing a method, according to an embodiment of the present disclosure.

### DETAILED DESCRIPTION

The terms “a” or “an”, as used herein, are defined as one or more than one. The term “plurality”, as used herein, is defined as two or more than two. The term “another”, as used herein, is defined as at least a second or more. The terms “including” and/or “having”, as used herein, are defined as comprising (i.e., open language). Reference throughout this document to “one embodiment”, “certain embodiments”, “an embodiment”, “an implementation”, “an example” or similar terms means that a particular feature, structure, or characteristic described in connection with the embodiment is included in at least one embodiment of the present disclosure. Thus, the appearances of such phrases or in various places throughout this specification are not necessarily all referring to the same embodiment. Furthermore, the particular features, structures, or characteristics may be combined in any suitable manner in one or more embodiments without limitation.

Wound care is a complex and broad topic in medicine with enormous associated costs, both economic and for patient healing outcomes and quality of life. Though numerous wound care products are available, most conform poorly to irregular wound surfaces and require a layer of adhesive coating for fixation, leading to difficult and painful application, with increased risk for epidermal desquamation during dressing changes. These shortcomings contribute to delays in wound healing and leave wounds susceptible to infection or scar formation, which can lead to complications ranging from disability or even death from fluid loss or sepsis.

Successful wound healing can benefit from adequate vascularity, clearance of dead tissue, prevention of infection, and appropriate moisture. After debridement, wound dressings can optimize these conditions by eliminating dead space, absorbing drainage and inflammatory wound exudate, and discouraging bacterial overgrowth. Additionally, the dressings can be reasonably affordable and simple to apply. Unfortunately, some pre-packaged wound dressings ranging from thin polymer films to alginate hydrogels often include significant trimming to fit awkward wounds, and frequent manipulation or reinforcement with tape or pressure dressings due to poor adhesive properties. This can be not only time-consuming but involve manual handling of the dressing and wound surfaces, which can introduce foreign bacteria.

Ill-fitting dressings can result in an increased frequency of dressing changes, leaving wounds exposed to foreign bacteria. This can lead to patients experiencing extended periods of pain. This can be especially true for very large wounds that call for twice daily dressing changes that take upwards of one hour for each treatment. Current dressings conform poorly to irregular wound shape and can require secondary adhesives for adequate fixation, leading to difficult and painful wound treatments, especially in children and adults with large wounds. These challenges in wound care result in delayed healing, with susceptibility to infection, scarring, and poor functional outcome. In the worst cases, inadequate wound care can result in sepsis, significant disability or even death. Patients with burn wounds covering greater than 40% total body surface area have a 75% risk of mortality due to infection. An observational cohort study (N=958) found that wound healing was achieved only in 53.3% of cases and the mortality rate was 9.4%, underscoring that current wound treatment procedures are suboptimal.

In certain populations, namely pediatric or severely wounded patients, dressing changes can be extensive and painful to an extent requiring sterile operating room conditions whilst under general anesthesia. Each dressing change, though beneficial for wound assessment, can increase the risk of infection and inevitably disrupts healing epidermis. This underscores the advantage of sprayable polymer-based wound dressing techniques that can result in: 1) reduced time required for dressing, 2) reduced pain due to minimum physical contact and eliminating the painful touch, 3) improved dressing conformation to irregular surfaces, and 4) improved healing time through less infection and irritation.

An ideal wound dressing would be easy and painless to apply, discourage microbial overgrowth, maintain a moist wound environment, and require infrequent dressing changes while still protecting the wound. Thus, solution blow spinning (SBS), or air-jet spinning, can be a solution to the aforementioned wound treatment issues. SBS can be used to deposit materials, such as fibers, for biomedical and electronics applications via a polymer (or other) solution and pressurized gas. The fabrication process for fibers can include concentric fluid streams, such as a stream for the polymer dissolved in a solvent and a stream for the gas, blown through concentric chambers of a nozzle. Generally, the gas can be blown through an outer chamber while the fluid is blown through an inner chamber, and the pressurized gas helps facilitate the deposition of the fluid onto a surface which forms fibers (of the polymer/material) upon evaporation of the solvent.

One example method for wound care includes electrospinning (e-spinning). Although the nanofibers prepared with e-spinning technology have been used in biomedicine, the technique has several limitations. E-spinning utilizes specialized high-voltage equipment, high-temperature, and intrinsically conductive polymers to spin nanofiber mats, and is often limited by slow fiber deposition. These factors not only raise safety and equipment process concerns, but also restrict the types of polymer and adjunctive therapies that can be used with this technology.

In contrast to skin grafting, epidermal cellular seeding with ASCS is less morbid and allows a small piece of skin to be used to cover a substantially greater surface area (1:80 expansion ratio, compared to traditional ratios of 1:1 to 1:4 with maximal meshing occurring at 1:6 in cases of extremely limited donor site availability). Multiple studies have shown that using sprayed skin cells in the form of ASCS on wounds is a viable, less invasive adjunct to STSG, particularly for patients whose wounds cover a very large surface area. ASCS is an accepted stand-alone treatment for deep partial-thickness burns as well as an adjunct to STSG in full-thickness burns, with the benefit of significantly reduced donor site wound size. The currently available ASCS preparation kit (RECELL® System, AVITA Medical, Valencia, CA) has been validated to produce viable skin cells, but the included spray nozzle attaches to a standard 10 mL syringe with a tendency to drip solution during application, leading to uneven spray rate and distribution of cells on the wound surface.

To this end, SBS is a low-cost technique that allows for rapid in situ deposition of polymer fibers via a simple airbrush device. The process is completed at ambient temperature and has been shown to have enhanced cell infiltration and proliferation when compared with e-spun fiber scaffolds. As SBS fiber mats are formed while spraying on the wound, they have the potential to be used as customizable wound dressings by altering the polymer formulation and can even be sprayed with adjunctive therapies at the time of deposition. Polymer composition can be selected to suit both low and high impact wound models via biodegradation, tissue adhesion, and flexibility, eliminating the need for non-degradable and cytotoxic secondary adhesives deployed in clinical settings.

As previously mentioned, autologous skin cell suspensions (ASCS) are useful for treating burn wounds, including in combination with split thickness skin grafts (STSG) for full thickness injuries. Currently, ASCS and dressing application are performed in a stepwise fashion, which allows the ASCS to leak out of the wound bed.

Therefore, described herein is a SBS device configured to co-spray ASCS and a polymer for full thickness wounds to address the current wound care limitations. Further, the ASCS and polymer co-spray via the SBS device is evaluated for efficacy. The SBS device additionally provides an advantageous, hand-held packaging form factor that can use pressurized gas and reusable hardware with sterile, disposable components to provide a low-cost and efficient wound dressing method. The polymer spray-on dressing used also addresses many of the aforementioned shortcomings since the polymer spray-on dressing 1) conforms to wound bed irregularity without manual handling of the wound, 2) remains flexible, allowing for patient movement without loss of adhesion, 3) is capable of absorbing excess wound exudate due to its fibrous structure, and 4) biodegrades gradually, loosening over time to leave fragile healing wound beds intact. The polymers used also result in dressings that 1) undergo a body temperature mediated glass transition and conform to tissue at body-temperature (poly(lactic-co-glycolic acid), PLGA), and 2) adhere in moist wound environments (poly(lactide-co-caprolactone), PLCL), resulting in strong tissue adherence without the use of reactive chemistries and toxic secondary adhesives.

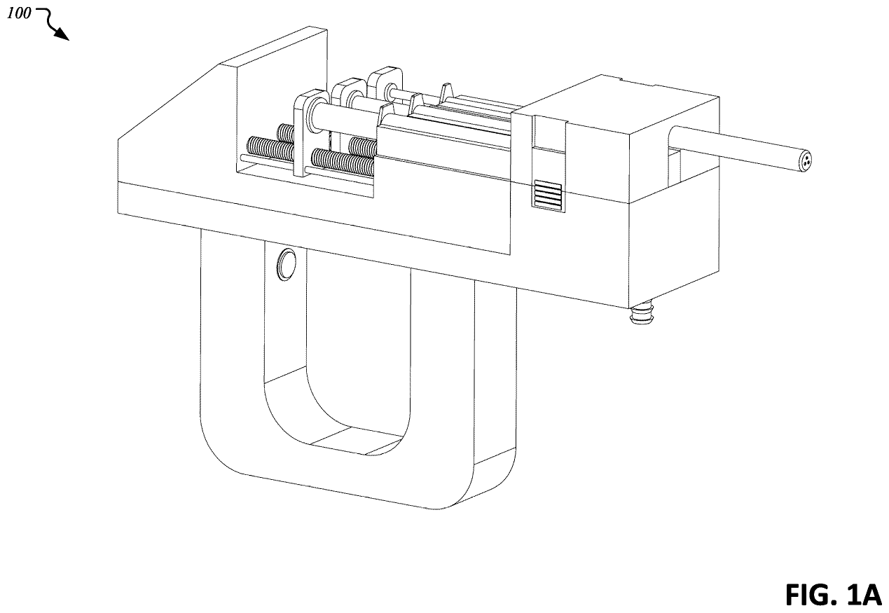

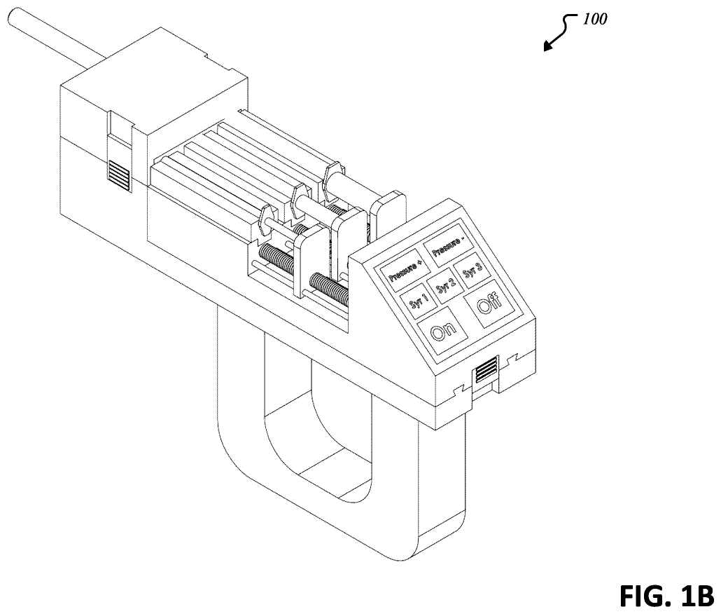

To this end, FIG. 1A and FIG. 1B are perspective view schematics of a blow spin device 100, according to an embodiment of the present disclosure. In an embodiment, as shown, the blow spin device blow spin device 100 presents a hand-held form factor with some features that can be re-used, while others can be disposed of and easily replaced with sterile parts for hygiene purposes. Notably, portions of the blow spin device 100 can be disassembled and reassembled for ease of replacing parts, storage, table-top use, etc.

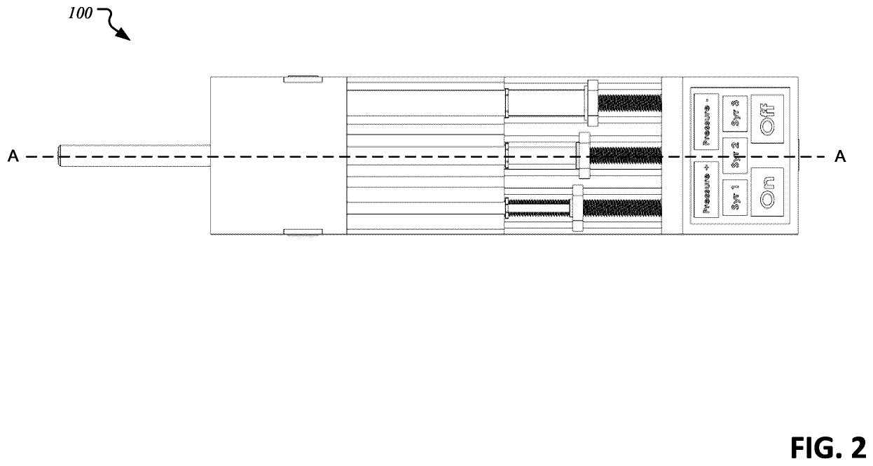

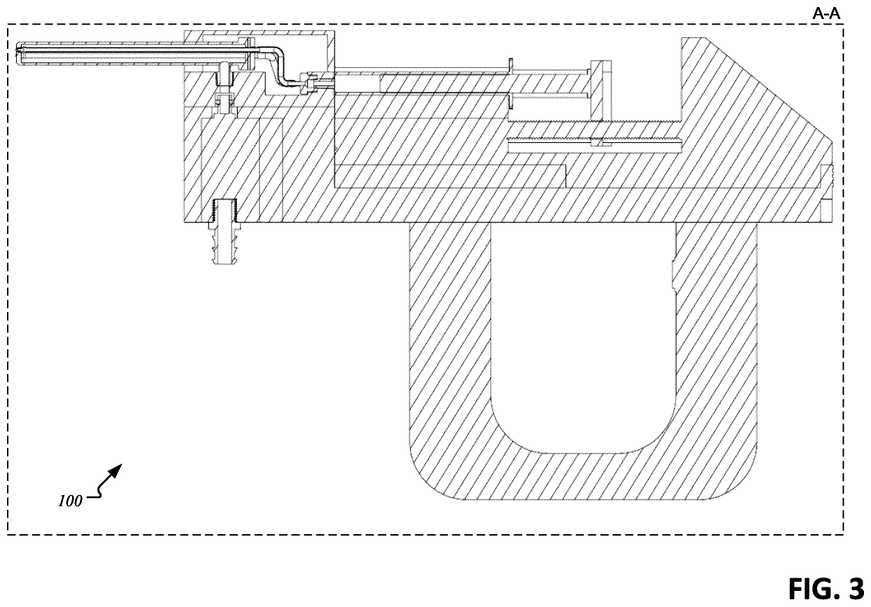

FIG. 2 is a top-down view schematic of the blow spin device 100, according to an embodiment of the present disclosure. In an embodiment, a cross-sectional plane A-A is shown through the blow spin device 100 and described in FIG. 3.

FIG. 3 is a cross-sectional schematic of the blow spin device 100 through the plane A-A, according to an embodiment of the present disclosure. In an embodiment, FIG. 3 highlights how the various features of the blow spin device 100 can be fluidly coupled to one another when assembled in order to simultaneously eject a solution with pressurized gas for fiber fabrication, wound dressing, etc.

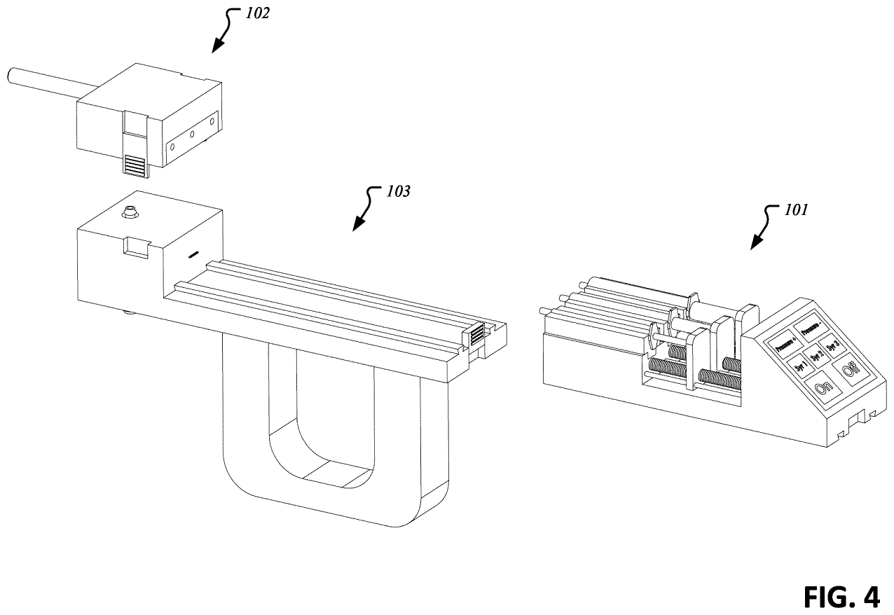

FIG. 4 is an exploded view schematic of the blow spin device 100, according to an embodiment of the present disclosure. In an embodiment, the blow spin device 100 includes a multi-syringe pump 101, a multi-nozzle airbrush adapter 102, and a handle base 103, and each part can be reversibly coupled or reversibly attached to one another to form the fully assembled blow spin device 100. For example, the multi-nozzle airbrush adapter 102 can be snap fit, friction fit, twist fit, fastened via screws, latches, toggles, pins, hook-and-loop (e.g., VELCRO), or bolts, or adhered to the handle base 103. As shown, the multi-nozzle airbrush adapter 102 includes tabs configured to reversibly snap fit onto complementary indentations on the handle base 103. Thus, the tabs of the multi-nozzle airbrush adapter 102 can be deformed to egress out of the complementary indentations of the handle base 103 in order to remove the multi-nozzle airbrush adapter 102 from the handle base 103. For example, the multi-syringe pump 101 can be coupled to the handle base 103 by sliding onto attachment rails along a first direction until abutting a stopping feature on the handle base 103, and optionally a retaining latch can be activated or engaged to prevent sliding of the multi-syringe pump 101 along a direction opposite the first direction.

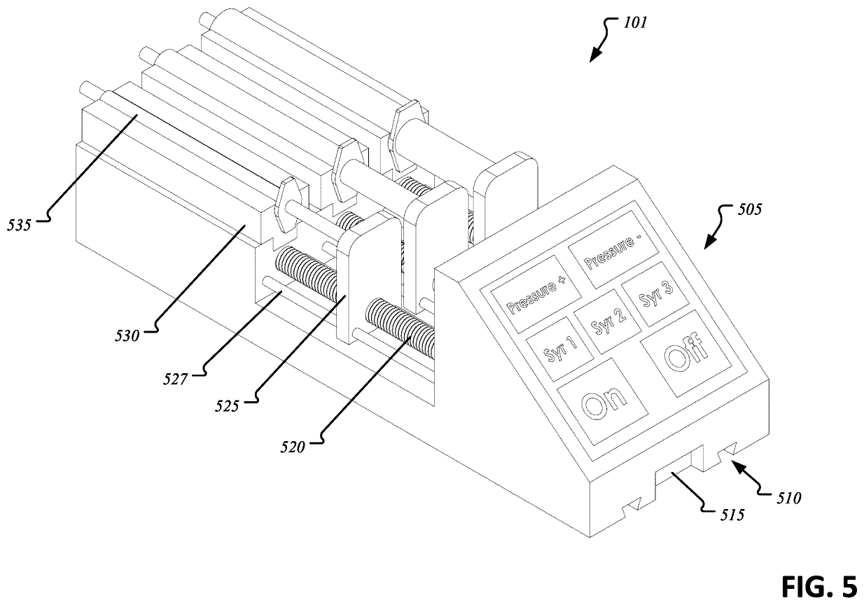

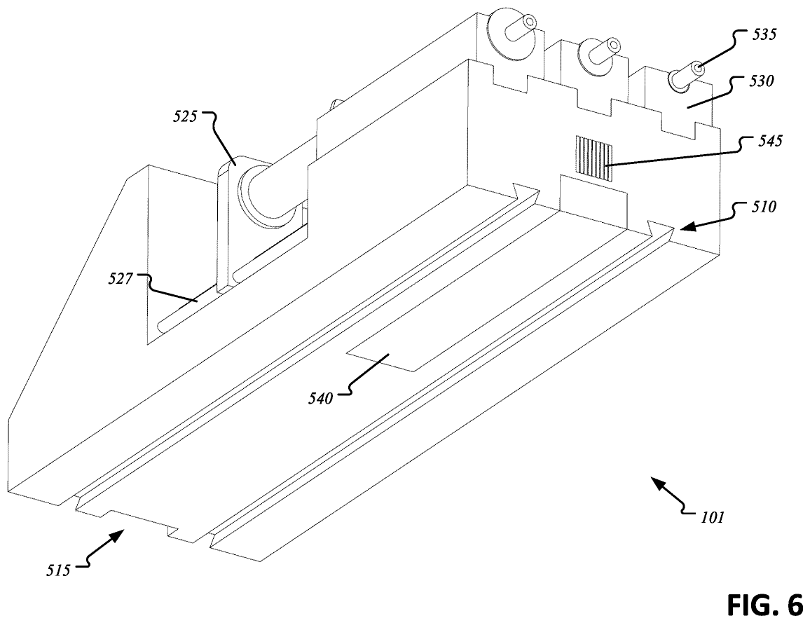

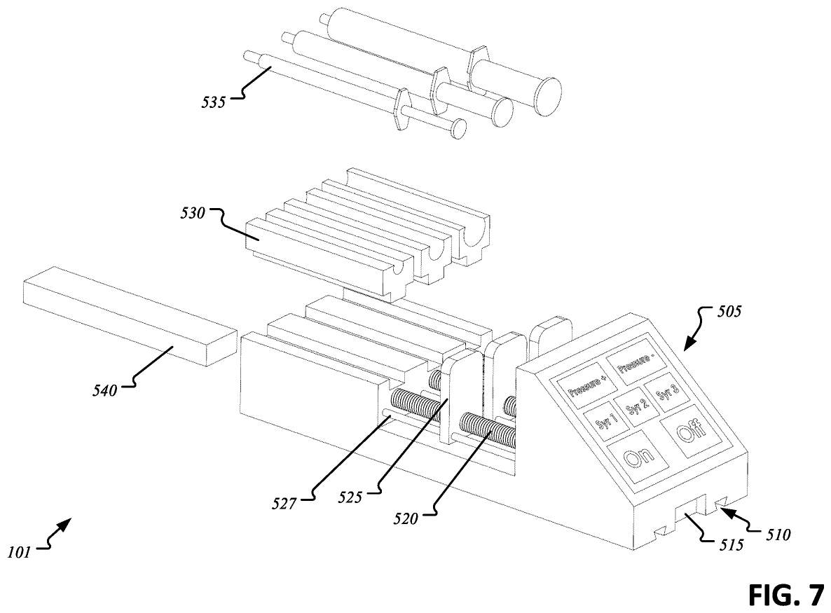

FIG. 5 is a perspective view schematic of the multi-syringe pump 101, according to an embodiment of the present disclosure. FIG. 6 is a perspective view schematic of the blow spin device 100 from an opposite angle, according to an embodiment of the present disclosure. FIG. 7 is an exploded view schematic of the multi-syringe pump 101, according to an embodiment of the present disclosure. In an embodiment, the multi-syringe pump 101 includes a parameter input interface 505, an alignment feature 510, a latching feature 515, a lead screw 520, a block 525, a rail 527, a syringe holder 530, a syringe 535, a battery 540, and a contact plate 545. In general, the multi-syringe pump 101 can be configured to hold one or more of the syringe 535 and adjust injection of a solution in the syringe 535. As shown, the parameter input interface 505 can be an area or surface on the multi-syringe pump 101 including an interface configured to receive an input, such as a parameter or setting input by a user. For example, the parameter input interface 505 can be a display configured to display one or more buttons to the user and each of the one or more buttons can adjust an operation or process of the multi-syringe pump 101. For example, the display can be an LCD display capable of sensing capacitive touch, a first button can turn on the multi-syringe pump 101, and a second button can turn off the multi-syringe pump 101. Additional buttons (whether physical or displayed via the display when the parameter input interface 505 is the display) can adjust, for example, selection of a syringe for modification, air pressure, flow rate, injection/extraction, pulse mode, or any combination of the aforementioned, among others. When the parameter input interface 505 is a display, the multi-syringe pump 101 can include processing circuitry electrically coupled to the parameter input interface 505 configured to process the input by the user and generate an instruction, such as an adjustment to an electrically coupled motor or actuator.

In an embodiment, the alignment feature 510 can be a shape disposed along a bottom surface of the multi-syringe pump 101 configured to align the multi-syringe pump 101 relative to the handle base 103. For example, the alignment feature 510 can be an elongated groove or cut-away disposed along a bottom surface of the multi-syringe pump 101 having a cross-sectional shape configured to engage with a rail having a complementary cross-sectional shape. As shown, the cross-sectional shape of the alignment feature 510 as the groove can be trapezoidal, and the complementary cross-sectional shape of the rail on the handle base 103 can also be trapezoidal (and slightly smaller in dimensions to allow the alignment feature 510 to fit over). The multi-syringe pump 101 can then slide onto the handle base 103 via the alignment feature 510 as the groove with the trapezoidal cross-sectional shape, which prevents the multi-syringe pump 101 from moving in any direction other than a direction of the sliding. While one of the alignment feature 510 as the groove is described, the multi-syringe pump 101 can include more than one of the alignment feature 510, such as two grooves, or more than two grooves. Of course, other cross-sectional shapes for the alignment feature 510 as the groove can be contemplated. In an embodiment, the alignment feature 510 can be one or more snap fit or friction fit shapes with complementary shapes on the handle base 103. For example, the alignment feature 510 can be a protrusion having a tip with a bulbous head configured to friction fit into a complementary depression or indent on the handle base 103. In particular, the depression or indent can narrow to form a neck towards the bottom of the indent through which the bulbous head of the alignment feature 510 can partially deform and then settle in a wider bottom of the indent, thereby providing a reversible coupling of the multi-syringe pump 101 to the handle base 103. Sufficient force can be applied to remove the multi-syringe pump 101 from the handle base 103, which can deform the head of the alignment feature 510 through the neck of the indent again.

In an embodiment, the latching feature 515 can be an indent or cut-out having a shape complementary to a latch (or similar fastening feature) on the handle base 103. The latching feature 515 can be disposed at an end of the multi-syringe pump 101 and configured to aid in reversibly fastening the multi-syringe pump 101 to the handle base 103. For example, the latch on the handle base 103 can be a spring latch configured to depress (deflect downwards) as the multi-syringe pump 101 slides onto the handle base 103 when the alignment feature 510 is the groove and, once the multi-syringe pump 101 slides fully onto the handle base 103, return (via the spring force) to a position where the latch is disposed in the latching feature 515, which can prevent the multi-syringe pump 101 from sliding out from the handle base 103. To remove the multi-syringe pump 101 from the handle base 103, the user can pull and depress the latch on the handle base 103, then slide the multi-syringe pump 101 off of the handle base 103. For example, the latch can have a spring-loaded lever action and a tip of the latch can have a hook, and the latching feature 515 can be an indent or cut-out having a shape complementary to the latch with a ledge for the hook to catch. When the latching feature 515 is a protrusion, the multi-syringe pump 101 can be pressed onto the handle base 103 and, when the protrusion of the multi-syringe pump 101 is arranged in the indent on the handle base 103, the latch can be disposed in the latching feature 515 and the hook can fully catch onto the ledge in the latching feature 515, which remains engaged due to the spring force. To remove the multi-syringe pump 101 from the handle base 103, the user can press on an opposite side of the lever of the latch to depress the spring, raise the hook of the latch out of the ledge of the latching feature 515, and pull the multi-syringe pump 101 away from the handle base 103.

In an embodiment, the lead screw 520 can be a threaded screw. The multi-syringe pump 101 can include one or more of the lead screw 520, and each of the one or more lead screw 520 can be coupled to an actuator or motor (not shown) disposed in or on the multi-syringe pump 101. The actuator can be, for example, a linear actuator electrically coupled to the parameter input interface 505 (and the processing circuitry).

The lead screw 520 can be coupled to the actuator at a first end of the lead screw 520, while a second end of the lead screw 520 can be fixed but free to rotate. As shown, the multi-syringe pump 101 can include three of the lead screw 520, and each of the lead screw 520 can be independently controlled. For example, the three lead screws 520 can each be controlled independently by a respective actuator. For example, the three actuators can be linear actuators as previously mentioned. In an embodiment, each of the linear actuators disposed on the multi-syringe pump 101 can be separately controlled by a microcontroller through a closed loop controller using linear/rotary encoders or linear/rotary potentiometers.

The actuator can be configured to rotate the lead screw 520 in a first rotational direction and a second rotational direction opposite the first rotational direction. Notably, any object coupled to the lead screw 520 can be translated along a length of the lead screw 520.

To this end, in an embodiment, the block 525 can be an object coupled to the lead screw 520 via a first opening in the block 525 having a complementary threading to the lead screw 520. When the lead screw 520 is rotated, the block 525 can be translated along a first direction parallel to the length of the lead screw 520, and when the lead screw 520 is rotated in the opposite rotational direction, the block 525 can be translated along a second direction parallel to the length of the lead screw 520. As will be described below, the block 525 can be configured to abut a plunger of a syringe and translate the plunger when the lead screw 520 is rotated.

In an embodiment, the block 525 can rotate with the rotation of the lead screw 520, which would result in no translation of the block 525. Therefore, the multi-syringe pump 101 can include the rail 527 disposed proximal to the lead screw 520, to which the block 525 can be additionally coupled. The block 525 can be coupled to the rail 527 via a second opening in the block 525. Here, the second opening can be smooth, and the rail 527 is configured to prevent rotation of the block 525 when the lead screw 520 is rotated. Thus, during rotation of the lead screw 520, the block 525 will be translated along the first direction parallel to the length of the lead screw 520 and the second direction parallel to the length of the lead screw 520.

In an embodiment, the multi-syringe pump 101 can include a platform portion disposed at an end of the multi-syringe pump 101 opposite the parameter input interface 505, and the platform portion can be configured to hold the syringe holder 530. The syringe holder 530 can be reversibly attached to the platform portion of the multi-syringe pump 101 and configured to hold the syringe 535. Thus, the syringe holder 530 can include a depression or indent for holding the syringe 535, and the depression or indent can have a shape complementary to a shape of the syringe 535. Notably, the syringe holder 530 can actively and reversibly secure the syringe 535 to the syringe holder 530 via friction fit or snap fit. Although not shown, the syringe 535 can have a clamshell shape in order to close overtop the syringe 535 for additional security. The clamshell shape can be secured when closed via, for example, a fastener, such as a latch, toggle, hook-and-loop, etc. Advantageously, the multi-syringe pump 101 can include one or more of the syringe holder 530, and each of the syringe holder 530 can be customized to the syringe 535 that the syringe holder 530 will hold. As shown, the multi-syringe pump 101 can hold three of the syringe 535, each having a different size or form factor. A material of the syringe holder 530 can be, for example, rubber, silicone, a polymer, a metal, or a composite.

In an embodiment, the syringe 535 can be a tube or cylindrical body with a tip or nozzle at a first end and an opening at a second end for receiving a plunger or piston. The opening at the second end of the syringe 535 can also include a flared flange. The flared flange can abut the syringe holder 530 when the syringe 535 is arranged in the syringe holder 530. The syringe 535 can be configured to hold a fluid in the tube and eject the fluid when the plunger is translated through the opening towards the tip of the syringe 535.

In an embodiment, the battery 540 can be electrically coupled to the parameter input interface 505 and/or the actuator and provide power for the parameter input interface 505 and the actuator. Additionally or alternatively, the multi-syringe pump 101 can be electrically connected to an external power source.

In an embodiment, the contact plate 545 can be an electrical contact configured to couple with a contact plate on the handle base 103 and mate the electrical components of the multi-syringe pump 101 with the multi-nozzle airbrush adapter 102 and/or the handle base 103.

In an embodiment, with a broader view of the multi-syringe pump 101, a starting configuration of the multi-syringe pump 101 can include the block 525 translated away from the platform portion, which can occur via actuation of the lead screw 520. Further, the syringe 535 can be partially or fully filled with a solution and arranged in the syringe holder 530 with the plunger of the syringe 535 partially or fully extended out towards the block 525 based on the fill level of the solution in the syringe 535. Thus, the tip of the syringe 535 is disposed at an opposite end at an edge of the multi-syringe pump 101. For example, the solution can be a polymer solution composed of 10% (w/v) PLGA with viscosity of 0.93 IV in acetone. For example, the solution can be a biocompatible polymer. For example, the biocompatible polymer at least one selected from the group consisting of poly(lactic-co-glycolic acid), poly(ethylene glycol), and poly(lactide-co-caprolactone). Notably, when the syringe 535 includes the flared flange, the syringe 535 can be arranged such that the flared flange abuts a surface of the syringe holder 530. It may be appreciated that a biomaterial is a substance or material that has been engineered to interact with biological systems for a medical purpose, such as a therapeutic or diagnostic goal.

In an embodiment, when the multi-syringe pump 101 transitions to an operation configuration due to, for example, an input by the user, the input via the parameter input interface 505 can actuate the actuator or motor to rotate the lead screw 520 at a set rate (rotation, speed, corresponding flow rate, corresponding pressure, etc.) in order to translate the block 525 to abut the plunger of the syringe 535 and translate the plunger. When the plunger is translated towards the tip of the syringe 535, the solution in the syringe 535 can be forced out of the syringe 535. The flared flange of the syringe 535 can prevent the syringe 535 from slipping in the syringe holder 530 and being forced out of the syringe holder 530 due to the force of the block 525 on the plunger. As shown, the multi-syringe pump 101 can include multiple of the syringe 535 and each of the syringe 535 can be set to a different setting. Additionally or alternatively, each of the syringe 535 can be actuated simultaneously, sequentially, or in any combination of tandem actuation.

In an embodiment, various setting or parameter inputs via the parameter input interface 505 can be adjusted based on the size of the syringe 535. For example, the syringe holder 530 being configured to hold a predetermined size of the syringe 535 can be detected by the multi-syringe pump 101. For example, the syringe holder 530 can include an identifying feature, such as a barcode, a quick-response (QR) code, RFID tag, unique fastening feature, or the like that can be detected by the multi-syringe pump 101. For example, the syringe holder 530 includes a barcode and the multi-syringe pump 101 includes a barcode scanner disposed proximal to the syringe holder 530 configured to read the barcode. For example, the syringe holder 530 includes a QR code and the multi-syringe pump 101 includes an imaging device (e.g., a camera) configured to obtain an image of the QR code. For example, the syringe holder 530 includes a RFID tag and the multi-syringe pump 101 includes an RFID reader configured to detect and identify the RFID tag using radio frequency/waves.

In an embodiment, the identity of the syringe holder 530 can result in an adjustment of the multi-syringe pump 101 during operation. For example, when the syringe holder 530 is configured to hold a 1 mL syringe 535 and the multi-syringe pump 101 detects this feature, the user input can translate to a different actuator response or operation of the lead screw 520 compared to when the syringe holder 530 is configured to hold a 20 mL syringe 535. That is, a same actuator response/operation to depress the plunger of the smaller syringe 535 the same distance as the larger syringe 535 will result in a greater volume of solution ejected from the larger syringe. Therefore, based on the identity of the syringe holder 530, the multi-syringe pump 101 (and the processing circuitry included therein) can adjust the actuator response/operation. For example, to eject a same volume of the solution from the smaller syringe 535, the actuator can be operated to depress the plunger of the smaller syringe 535 farther/faster/a greater distance compared to the actuator of the larger syringe 535.

In an embodiment, the syringe holder 530 can be configured to hold an object besides a common syringe (tube and long plunger), such as a cartridge holding a solution. For example, the cartridge can include a prepared solution in a sealed, sterile container, while including a tip, cylindrical body, and plug at an end opposite the tip of the cartridge. The plug can be, for example, a short plunger, or a film configured to seal the opening of the cartridge opposite the tip. Thus, to eject the solution from the cartridge via the tip, the block 525 can include a rod configured to insert into the end opposite the tip of the cartridge and push the solution towards the tip of the cartridge. For example, when the plug is a short plunger, the rod can abut and depress the short plunger. For example, when the plug is a sealing film, the rod can abut and break the sealing film around the opening, and then immediately seal itself (the rod) against sidewalls of the cylindrical body. The rod (and the block 525) can thus become the plunger.

Advantageously, this can allow for additional ease of replacement of not just the syringe 535, but also the syringe holder 530 when the syringe 535 (cartridge) and syringe holder 530 are packaged together. Notably, the syringe holder 530 can include the identifying feature that also identifies the contents of the solution in the cartridge. A corresponding rod can be packaged with and used with the cartridge when the block 525 includes a feature for installing and replacing various rods. Of course, as previously described, the syringe 535 can also be swapped or replaced, and therefore the syringe 535 being a common syringe can similarly be packaged with the syringe holder 530. However, the common syringe holding a freshly prepared solution in a non-sterile environment can potentially allow contamination or spoiling of the solution when the solution is not used immediately and stored long-term without sterilization, whereas a cartridge prepared and sealed in a sterile environment can allow longer shelf life until used.

Furthermore, based on the syringe 535 and the solution, various spray profiles can be defined. Here, spray profile is defined as a setting for the ejection of the solution from the syringe 535, or the injection of the solution into the multi-nozzle airbrush adapter 102. This can be, for example, the previously described flow rate. In an embodiment, the various spray profiles can be defined for each actuator to control a spray (ejection/injection) for each of the solutions. For example, when the multi-syringe pump 101 includes three of the syringes 535, three different spray profiles can be used to individually control each actuator and therefore spray each of the three solutions differently. The spray profiles can be defined by, for example, the user inputting settings via the parameter input interface 505. As shown in FIG. 5, in a non-limiting example, the parameter input interface 505 can include a button to select one of the three syringes 535 (“Syr 1,” “Syr 2,” and “Syr 3”) for adjusting the spray profile (or using a unique button press length or pattern to enter a mode to adjust the profiles). The user can press the “Syr 1” button, which sets the spray profile for a first syringe as the active profile for adjusting, after which the user can increase or decrease a desired pressure using the “Pressure +” or “Pressure −” buttons. Of course, the “Pressure +” or “Pressure −” buttons can be configured (and labeled accordingly) to adjust a different setting, such as flow rate, screw speed, etc. When the parameter input interface 505 is a display, the user can cycle through various different settings for adjusting the spray profile.

In an embodiment, the user can adjust the spray profile to a desired setting, and the processing circuitry can process the input and set the actuator for each respective syringe accordingly. For example, the user can set the first syringe to eject solution at 1 mL/min and the processing circuitry can determine the respective actuator rotation speed to rotate the lead screw accordingly to translate the block 505 accordingly to depress the plunger in the 1 mL syringe at 1 mL/min. As previously described, the size of the syringe 535 will also be identified and set in the spray profile. Further, when there are multiple of the syringe 535, the user can set a profile for each of the syringes. The spray profile can also include a spray modality that how the solution is sprayed. For example, the spray can be continuous. For example, the spray can be pulsed at repeating intervals. For example, the spray can be pulsed at irregular intervals set by the user.

In an embodiment, the spray profiles can be set via an app or program on a connected device. For example, the multi-syringe pump 101 can be connected to a computer and a program on the computer can be used to configure the multi-syringe pump 101 and the spray profiles. The connection can be wired, such as via ethernet, USB, other tethering, etc. and can be permanent or via an as-needed pair/sync to update the profiles. For example, the multi-syringe pump 101 can be connected to a wireless device, such as a smart phone, and the wireless device can be used to configure the multi-syringe pump 101 via the app. The connection between the multi-syringe pump 101 and the wireless device can be, for example, Wi-Fi, Bluetooth, 5G or other cellular, NFC, infrared, etc., The Wi-Fi connection can be via, for example, an esp32 microcontroller Wi-Fi module.

In an embodiment, the spray profiles can be received from a server storing a number of pre-set spray profiles. The spray profiles on the server can be varied based on the solution and syringe 535 and desired spray and spray modality. The options can be automatically obtained/retrieved/requested and presented upon scanning of the identifying feature of the syringe 535 or the syringe holder 530. The spray profiles can be obtained via the computer or smart phone or communicatively coupled device, or directly via the blow spin device 100.

Notably, in each embodiment, the user can set the spray volume and spray modality for each syringe. The user can also set the spray volume and spray modality more generally for all of the syringes during the entire operation length to generate an operation profile. For example, the user can set the first syringe to spray continuously at 0.1 mL/min, a second syringe to spray at 0.1 mL/min every 0.5 seconds with a 0.25 second break with no spray, and a third syringe to spray at 0.2 mL/min every 0.1 seconds with a 0.1 second break. The multi-syringe pump 101 (and processing circuitry) can then automatically process the desired settings and operate each actuator individually to achieve the desired spray (flow rate) and spray modality.

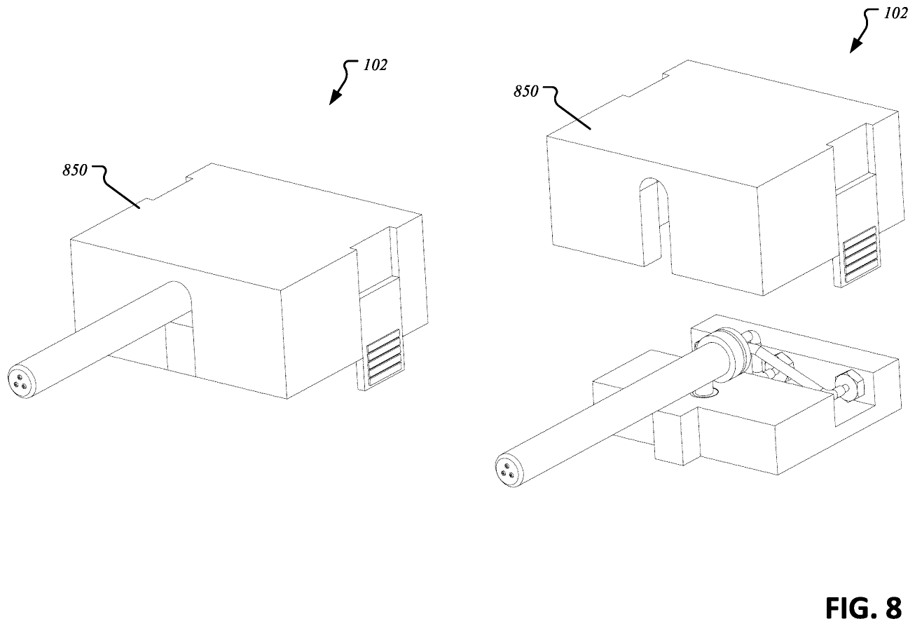

FIG. 8 is a perspective view schematic of the multi-nozzle airbrush adapter 102, according to an embodiment of the present disclosure. In an embodiment, the multi-nozzle airbrush adapter 102 can include a housing 850 for covering internal components. The housing 850 can be reversibly attached to a base holding the internal components. As shown, the housing 850 can include locking tabs.

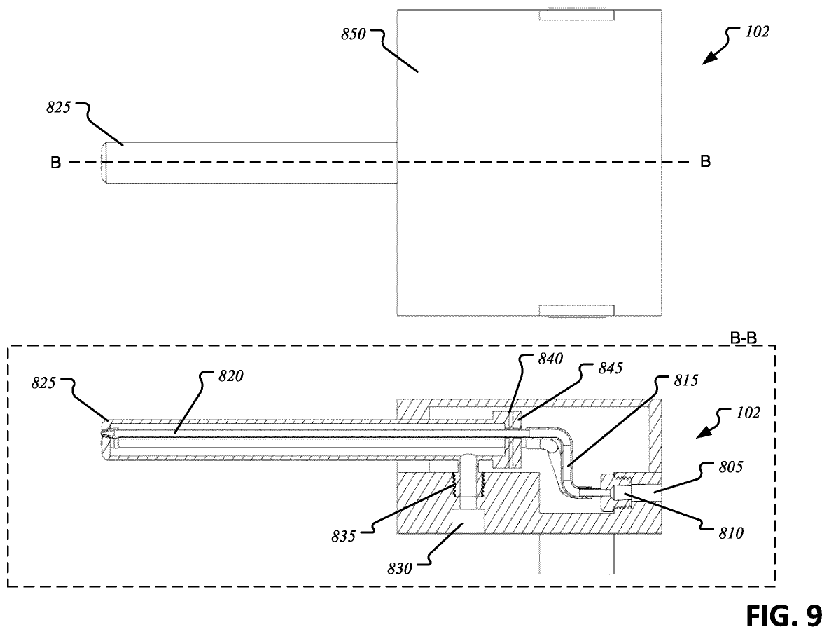

FIG. 9 is a top-down and cross-sectional view schematic of a multi-nozzle airbrush adapter, according to an embodiment of the present disclosure. In an embodiment, a cross-sectional plane B-B is shown through the multi-nozzle airbrush adapter 102. The multi-nozzle airbrush adapter 102 can include a syringe fitment 805 (or nozzle), a solution channel adapter 810, a solution channel tubing 815, a needle 820, a body 825, an air channel inlet 830, an air channel adapter 835, a gasket 840, and a rear cap 845. The base of the multi-nozzle airbrush adapter 102 can include a first portion including the syringe fitment 805 and the solution channel adapter 810, an open portion including the solution channel tubing 815, and a second portion including the body 825 coupled thereto and the air channel inlet 830.

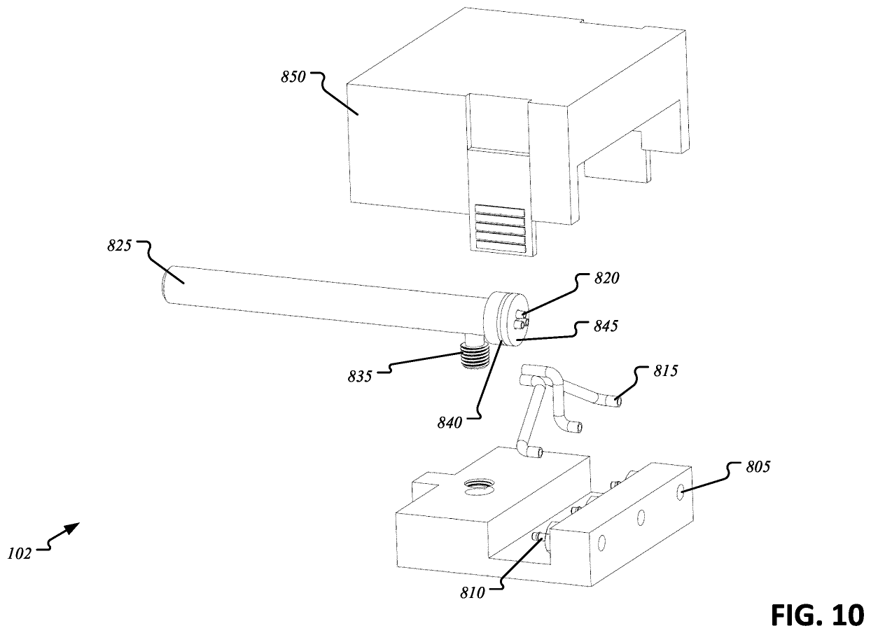

FIG. 10 is an exploded view schematic of the multi-nozzle airbrush adapter 102, according to an embodiment of the present disclosure. In an embodiment, the multi-syringe pump 101 can be fluidly coupled to the multi-nozzle airbrush adapter 102. When the multi-syringe pump 101 and the multi-nozzle airbrush adapter 102 are coupled, the tip of the syringe 535 can be arranged in the syringe fitment 805. That is, the platform portion of the multi-syringe pump 101 can abut a first side of the first portion of the base of the multi-nozzle airbrush adapter 102. The syringe fitment 805 can be an opening configured to receive the tip of the syringe 535 at the first side of the first portion and any solution injected by the syringe 535 into the syringe fitment 805. The syringe fitment 805 can have a shape designed to optimally receive the tip of the syringe 535 to form a fluid seal and reduce any leakage from the connection point. For example, the syringe 535 can be a Luer syringe with a Luer slip tip, and the syringe fitment 805 can be designed as a female Luer slip tip adapter. The syringe fitment 805 can extend through the first portion of the multi-nozzle airbrush adapter 102 to a second side of the first portion. The solution channel adapter 810 can be disposed at the second side of the first portion and coupled to syringe fitment 805 at the second side of the first portion. For example, the syringe fitment 805 at the second side of the first portion can be threaded and the solution channel adapter 810 can have a first end including complementary threading. The solution channel adapter 810 can be threaded into the syringe fitment 805. Additionally or alternatively, the solution channel adapter 810 can be coupled to the syringe fitment 805 via snap fit or friction fit. The solution channel adapter 810 can be hollow and a second end of the solution channel adapter 810 can be configured to couple with the solution channel tubing 815 via snap fit or friction fit. Thus, the second end of the solution channel adapter 810 can be disposed in the open portion of the base, along with the solution channel tubing 815.

In an embodiment, the solution channel tubing 815 can be hollow tubing configured to carry any solution injected by the syringe 535 through the syringe fitment 805, into the solution channel adapter 810, and into the solution channel tubing 815. The solution channel tubing 815 can be flexible or rigid. Notably, the solution channel tubing 815 can be removed and replaced if needed. Similarly, the solution channel adapter 810 can be removed and replaced if needed. Furthermore, different form factors for the solution channel adapter 810 and the solution channel tubing 815 can be used based on desired parameters, such as flow rate. The solution channel tubing 815 can be fluidly coupled to the needle 820 disposed in the body 825.

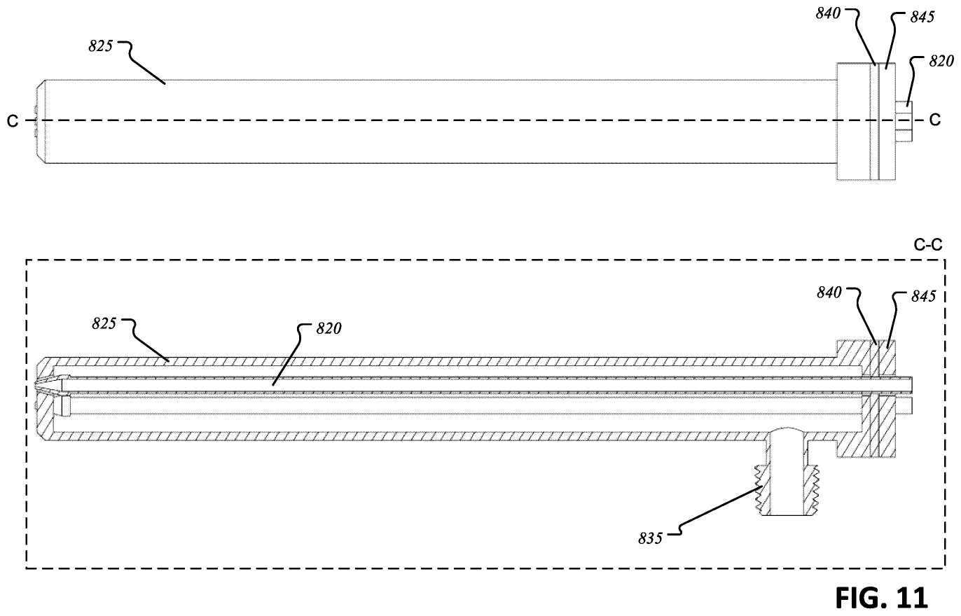

To this end, FIG. 11 is a top-down and cross-sectional view schematic of the body 825, according to an embodiment of the present disclosure. In an embodiment, a cross-sectional plane C-C is shown through the body 825. The solution channel tubing 815 can be fluidly coupled to a rear of the needle 820 at an end opposite a tip of the needle 820. As shown, the needle 820 can be arranged in a hollow cavity of the body 825 and extend a length of the needle 820. The body 825 can thus be a hollow tube with the tip having an opening or through hole for the tip of the needle 820. The needle 820 can be a hollow tube as well and configured to receive the solution injected from the syringe 535. The tip of the needle 820 can protrude through a tip of the body 825 (through the through hole) at an end of the body 825 opposite the rear cap 845. Notably, the tip of the needle 820 is spaced from the body 825 at the through hole in order to allow gas to flow between the tip of the needle 820 and the body 825. In such a design, the concentric streams for the solution and the pressurized gas are realized. Advantageously, the body 825 can house more than one of the needle 820 to form the multiple nozzles of the multi-nozzle airbrush adapter 102. Each needle can have its own corresponding through hole at the tip of the body 825. A material of the needle 820 can be, for example, stainless steel.

In an embodiment, an outer diameter of the body 825 along the hollow tube can be, for example, less than 20 mm, or less than 15 mm, or less than or equal to 12 mm. When the outer diameter of the body 825 is less than or equal to 12 mm, the body 825 can be compatible or accepted for certain medical procedures where a trocar is needed for access. For procedures with smaller trocars, a single nozzle or needle 820 can be used with a narrower diameter of the body 825. A length of the body 825 can be adjusted as well. For example, a longer length for the body 825 can be used to facilitate spraying during a laparoscopic procedure. A longer length of the body 825 can also be beneficial for external wound dressing. The body 825 and the needles 820 can come in various predetermined sizes and shapes compatible with the multi-nozzle airbrush adapter 102 in order to allow swapping of the body 825 and the needles 820 based on the application or procedure.

Referring back to FIG. 9, the pressurized gas can be introduced into the body 825 via the air channel inlet 830. In an embodiment, the air channel inlet 830 can be an opening disposed in the second portion of the base of the multi-nozzle airbrush adapter 102 extending through the entirety of the second portion. The body 825 can be coupled to the air channel inlet 830 via the air channel adapter 835. The air channel inlet 830 can be a female receptacle and the air channel adapter 835 can be a hollow male stem, and the air channel inlet 830 can be configured to receive the air channel adapter 835 therein. For example, the air channel adapter 835 can include external threading and the body 825 can be threaded into the air channel inlet 830 having complementary internal threading to secure the body 825 to the base of the multi-nozzle airbrush adapter 102. For example, the air channel adapter 835 can include external baffles and be push fit into the air channel inlet 830 to form a fluid seal. Upon coupling with the air channel inlet 830, pressurized gas can be introduced via the handle base 103 from an external source and enter into the hollow chamber of the body 825 through the air channel inlet 830 and the air channel adapter 835, after which the pressurized gas can exit through the space between the tip of the needle 820 and the tip of the body 825. To prevent leakage of the pressurized gas through the coupling point between the needle 820 and the body 825 at the end opposite the tip of the needle 820 and the body 825, the gasket 840 can be configured to receive the needle 820 inserted therein and fit snugly around the tube (shaft) of the needle 820. The gasket 840 can be disposed at the end of the body 825. A material of the gasket 840 can be, for example, rubber, silicone, polymer, or other known material for use in gaskets.

In an embodiment, the rear cap 845 can be arranged and the end of the body 825 after the gasket 840 has been arranged on the same end of the body 825. The rear cap 845 can be configured to receive the needle 820 inserted therein as well, and help secure the needle 820 and the gasket 840 to the body 825.

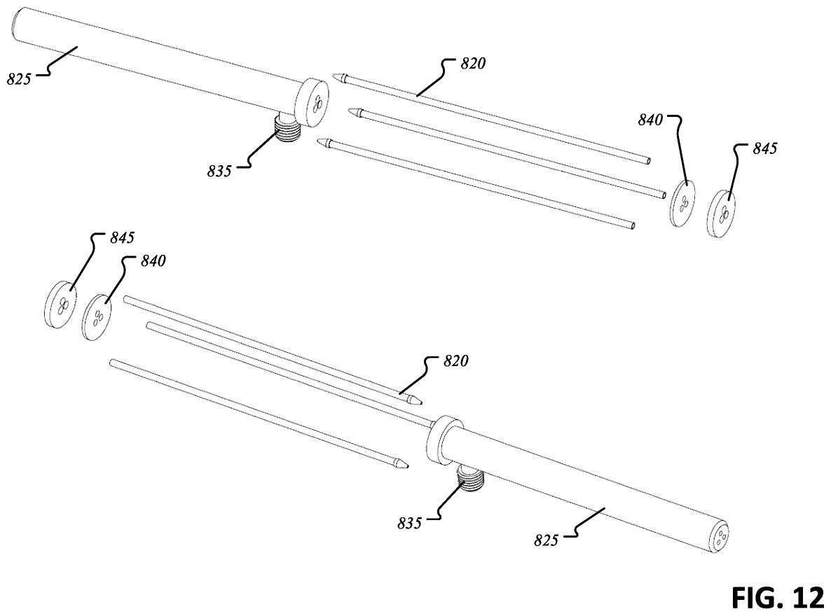

FIG. 12 is an exploded view schematic of the needle 820 of the multi-nozzle airbrush adapter 102, according to an embodiment of the present disclosure. In an embodiment, as shown, the multi-nozzle airbrush adapter 102 can include more than one of the needle 820, such as three of the needle 820, and each of the needle 820 can be configured to receive the injected solution from a corresponding syringe 535 filled with a unique solution. Furthermore, since the multi-syringe pump 101 can be adjusted to independently address each of the corresponding syringe 535, the flow through each of the needle 820 can be different. Advantageously, the multiple solutions from the multiple syringes can be injected simultaneously or separately. When injected separately, the gas can continue to flow and the various solutions can be injected immediately one after another, eliminating any delay caused by the need to swap out parts, actuators, or solutions, thus leading to a better wound dressing result.

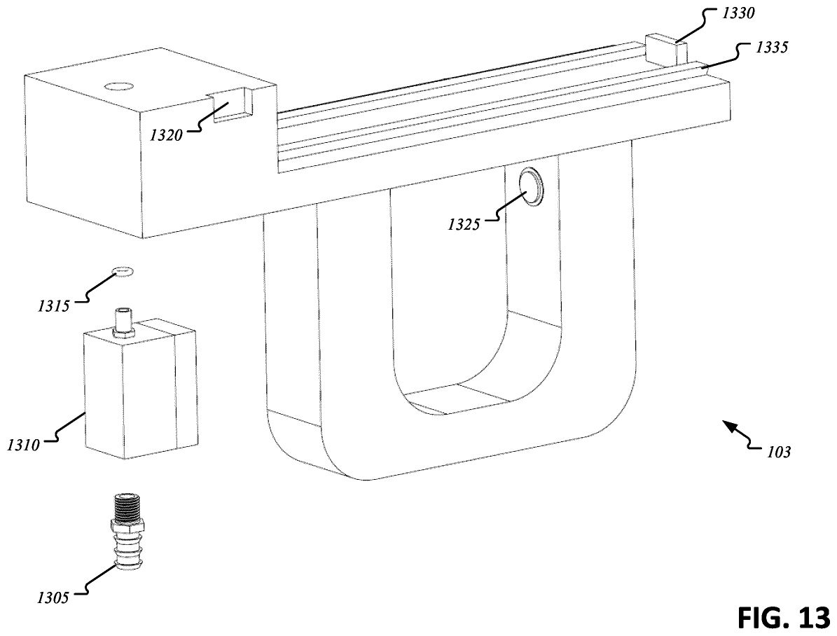

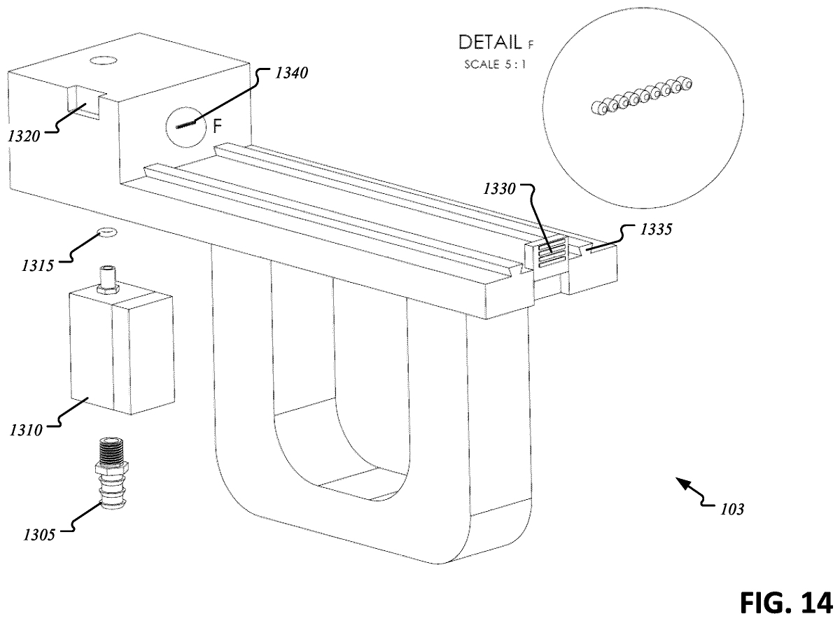

FIG. 13 is a perspective view schematic of the handle base 103, according to an embodiment of the present disclosure. FIG. 14 is a perspective view schematic of the handle base 103 at an opposite angle, according to an embodiment of the present disclosure. In an embodiment, the handle base 103 can include a barbed stem 1305, a pressure regulator 1310, an o-ring 1315, a latching feature 1320, a push button 1325, a latch 1330, a complementary latching feature 1335, and electrical pins 1340. The handle base 103 can include a handle for the user to grip, thereby providing the hand-held form factor. As shown, the handle is a U-shaped handle, which can allow for a one- or two-handed grip, where the two-handed grip can provide additional stability. The barbed stem 1305 can be coupled to the pressure regulator 1310, which can be coupled to a first end of the handle base 103 configured to couple with the multi-nozzle airbrush adapter 102. The barbed stem 1305 can be configured to connect to an external source of pressurized gas.

In an embodiment, the pressure regulator 1310 can be an electronic valve equipped with a standard thread to allow for configurable connections to the pressurized air. The o-ring 1315 can be configured to form a seal with an opening formed through the first end of the handle base 103 to which the pressure regulator 1310 is coupled.

In an embodiment, the latching feature 1320 can be a complementary female feature for securing the multi-nozzle airbrush adapter 102 to the handle base 103 via the male features (locking tabs) of the housing 850.

In an embodiment, the push button 1325 can be configured to initiate the air and/or the syringe actuator in a blow spinning application. When the handle base 103 is connected to the multi-syringe pump 101, the push button 1325 can be programmed to control both the pressure regulator 1310 and the dispensing of fluids.

In an embodiment, the latch 1330 of the handle base 103 can be the complementary male feature for securing the multi-syringe pump 101 to the handle base 103 as previously described. For example, the latch 1330 can be the spring latch. For example, the latch 1330 can be the spring-loaded lever having a hook shape or feature on one side of the lever for engaging with a ledge on the latching feature 515 of the multi-syringe pump 101.

In an embodiment, the complementary latching feature 1335 can be the complementary feature for aligning the multi-syringe pump 101 to the handle base 103 as previously described. For example, the complementary latching feature 1335 can be male alignment rails. For example, the complementary latching feature 1335 can be female indents or depressions with a narrowing neck feature towards the bottom of the complementary latching feature 1335.

In an embodiment, the electrical pins 1340 can be electrical pogo pins configured to connect with the electrical contacts of the contact plate 545 of the multi-syringe pump 101. Thus, when the multi-syringe pump 101 and the handle base 103 are coupled, the push button 1325 can be enabled to control both the pressure regulator 1310 and the dispensing of fluids.

Notably, the blow spin device 100 can be fabricated using a combination of reusable hardware and sterile, disposable spray nozzle components. The blow spin device 100 can be sterilized using low-temperature methods available in most medical facilities. A material for the disposable parts can be, for example, polypropylene. Polypropylene is a sterilizable, biostable, and biocompatible polymer used for medical syringes, sutures, and load-bearing implants.

### Experimental Results

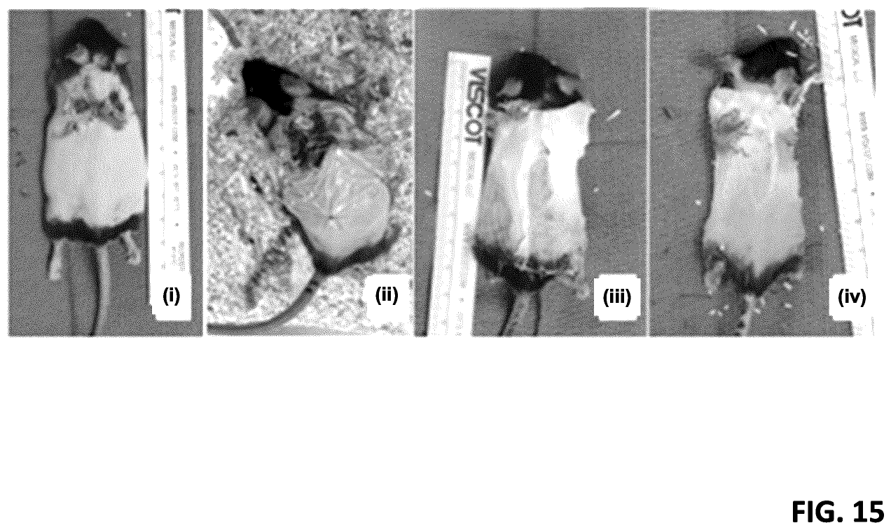

FIG. 15 shows images of mice wound dressing, according to an embodiment of the present disclosure. In panel (i), a mouse shown at day 0 after wounding/stenting. In panel (ii), a mouse is dressed with Tegaderm dressing. In panel (iii), a mouse is sprayed with the blow spin device 100. In panel (iv), a mouse is sprayed with a commercial airbrush.

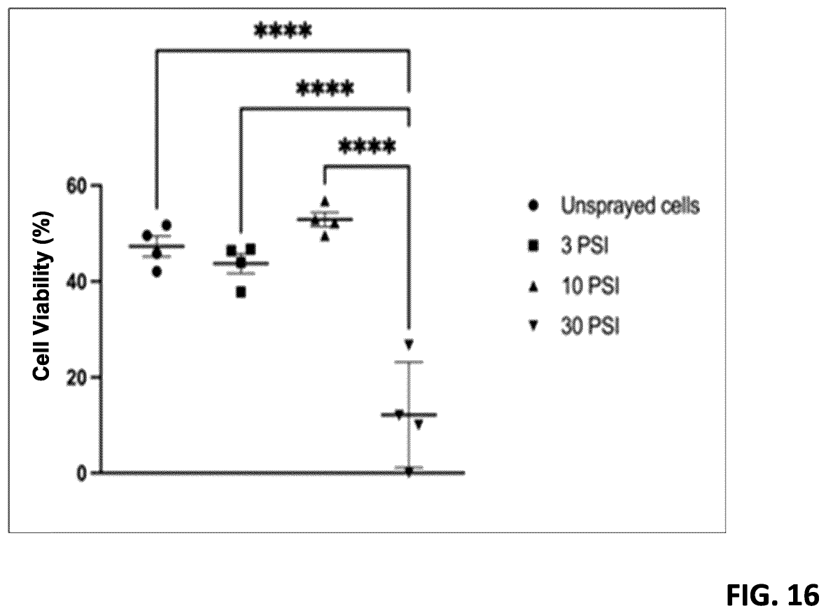

FIG. 16 is a plot describing cell viability as a function of spray pressure, according to an embodiment of the present disclosure. PLCL, PLGA, and mouse ASCS were sprayed and tested for 1) fiber formation when spraying polymer and 2) the effect of spraying and co-spraying on ASCS cell viability. A pressure regulator (1-30 psi), such as the pressure regulator 1310, was used to assess the effect of device air flow on fiber formation. Visible fiber formation was seen when ASCS was co-sprayed with polymer. No significant decreases in ASCS cell viability (assessed with a live/dead assay, Invitrogen, L3224) resulted with spraying at 3 and 10 psi, though viability was significantly decreased when ASCS was sprayed at 30 psi. Additionally, incubating ASCS with PLCL and PLGA polymers after spraying with the blow spin device 100 did not result in loss of cell viability. To assess the longer-term effects of spraying on cell viability, mouse melanoma B16 cells were sprayed using the device and cell viability was measured using trypan blue techniques immediately after spraying (0 hr) and at 24 hr and 48 hr. B16 cells were chosen as they are an established cell line of skin tissue origin that are known to survive in vitro, while ASCS cells are much more susceptible to cell death if not placed immediately onto a wound bed. No significant decreases in % viability was observed at any time point.

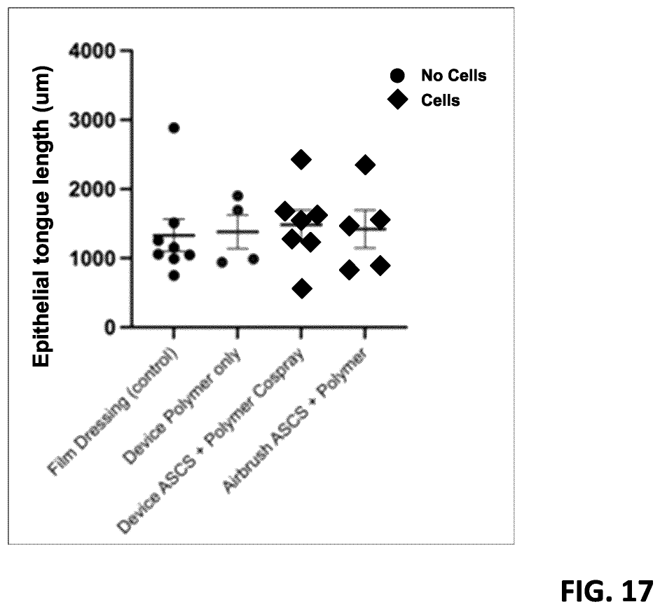

To this end, FIG. 17 is a plot describing epithelial tongue length for various dressings in a mouse model, according to an embodiment of the present disclosure. As shown, the ASCS and polymer co-spray using the blow spin device 100 yielded a longer average epithelial tongue length compared to film dressing, polymer spray only, and ASCS and polymer co-spray using an industrial airbrush. Notably, the industrial airbrush design as referenced previously does not accommodate customizable control of polymer feed rate, leading to polymer deposition rates without accurate control mechanisms aside form air pressure.

Therefore, advantageously, the blow spin device 100 as described combines the advantages of blow spinning technology and ASCS spraying. Therefore, the blow spin device 100 addresses a substantial technical gap by enabling simultaneous spraying of skin cells and SBS polymer, streamlining the treatment of partial thickness wounds. The blow spin device 100 is a medical-grade device that can simultaneously spray biodegradable polymer solutions and a patient's own skin cells to promote healing while providing a custom wound barrier.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more Such back-end, middleware, or front-end components. The components of the system can be interconnected by any form or medium of digital data communication, e.g., a communication network. Examples of communication networks include a local area network (LAN) and a wide area network (WAN), e.g., the Internet.

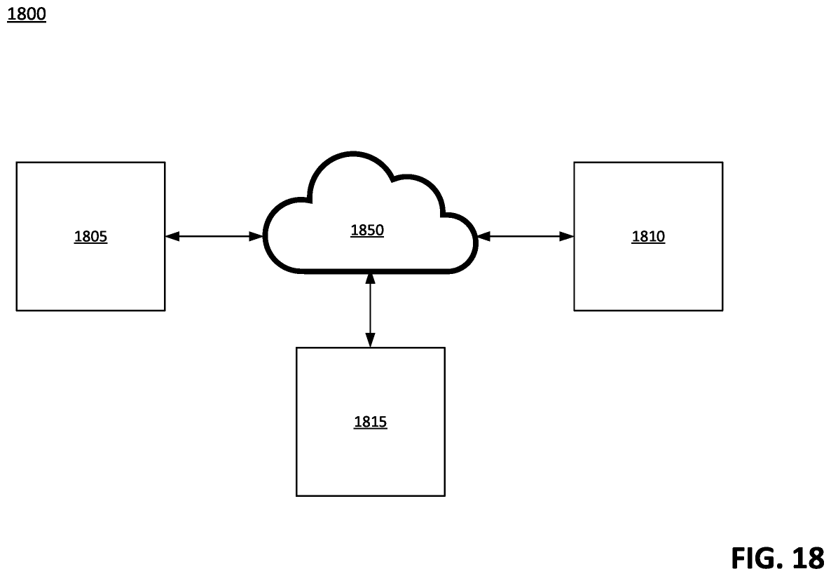

FIG. 18 is a schematic view of a system 1800, according to an embodiment of the present disclosure. In an embodiment, the system 1800 can include a first device 1805, such as the blow spin device 100, communicatively connected to a second electronic device 1810, such as a server, via a network 1850. A third electronic device 1815, such as a computer or smart phone, can be communicatively connected to the first device 1805 and the second electronic device 1810. The devices can be connected via a wired or a wireless connection. The connection between, for example, the first device 1805 and the second electronic device 1810 can be via the network 1850, wherein the network 1850 is wireless or wired. In an embodiment, the first device 1805 can be configured to obtain data from the user (of the first device 1805), such as an input relating to a parameter or setting of the multi-syringe pump 101. Notably, the first device 1805 can transmit the data over the communication network 1850 to the networked second electronic device 1810 and/or the third electronic device 1815.

In an embodiment, the first electronic device 1805 need not be communicatively coupled to the other device or the network 1850. That is, the method described herein can be run entirely on the first device 1805 using the obtained data.

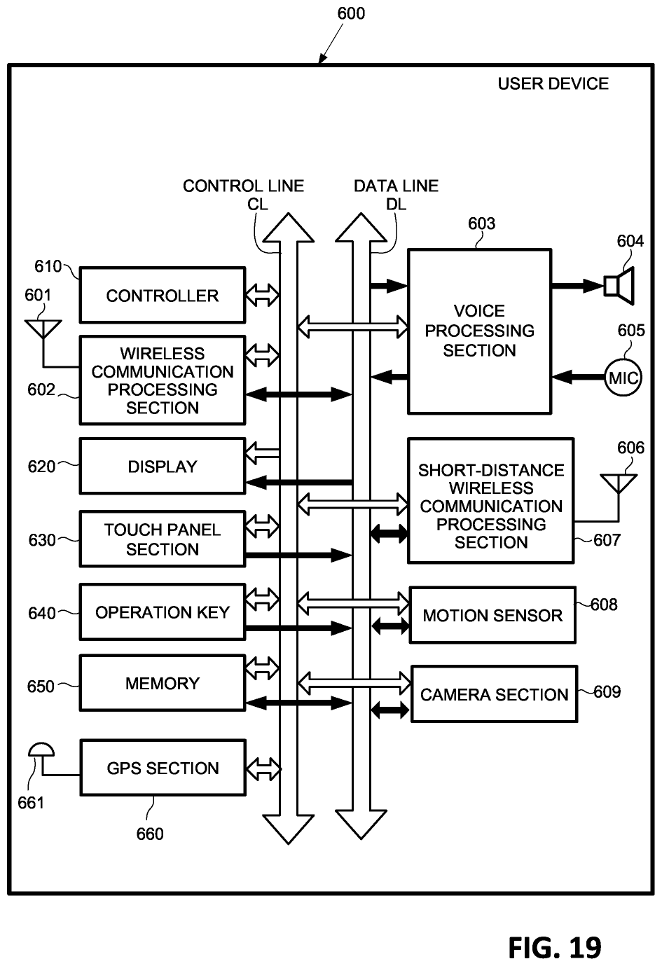

In an embodiment, the first device 1805 can include a central processing unit (CPU), among other components (discussed in more detail in FIGS. 19-21). An application can be installed or accessible on the first device 1805 for executing the methods described herein. The application can also be integrated into an operating system (OS) of the first device 1805. The first device 1805 can be or include integrated therein any electronic device such as, but not limited to, a smart-phone, a personal computer, a tablet pc, a smart-watch, a smart-television, an interactive screen, an IoT (Internet of things) device, or the like. Although the above description was discussed with respect to the first device 1805, it is to be understood that the same description applies to the other devices (1810 and 1815) of FIG. 18.

The computing system can include clients (user devices) and servers. A client and server are generally remote from each other and typically interact through a communication network. The relationship of client and server arises by virtue of computer programs running on the respective computers and having a client-server relationship to each other. In an embodiment, a server transmits data, e.g., an HTML page, to a user device, e.g., for purposes of displaying data to and receiving user input from a user interacting with the user device, which acts as a client. Data generated at the user device, e.g., a result of the user interaction, can be received from the user device at the server.

Electronic device 600 shown in FIG. 19 can be an example of one or more of the devices shown in FIG. 18. In an embodiment, the device 600 may be a smartphone. However, the skilled artisan will appreciate that the features described herein may be adapted to be implemented on other devices (e.g., a laptop, a tablet, a server, an e-reader, a camera, a navigation device, etc.). The device 600 of FIG. 19 includes processing circuitry, as discussed above. The processing circuitry includes one or more of the elements discussed next with reference to FIG. 19. The device 600 may include other components not explicitly illustrated in FIG. 19 such as a CPU, GPU, main memory, frame buffer, etc. The device 600 includes a controller 610 and a wireless communication processor 602 connected to an antenna 601. A speaker 604 and a microphone 605 are connected to a voice processor 603.

The controller 610 may include one or more processors/processing circuitry (CPU, GPU, or other circuitry) and may control each element in the device 600 to perform functions related to communication control, audio signal processing, graphics processing, control for the audio signal processing, still and moving image processing and control, and other kinds of signal processing. The controller 610 may perform these functions by executing instructions stored in a memory 650. Alternatively, or in addition to the local storage of the memory 650, the functions may be executed using instructions stored on an external device accessed on a network or on a non-transitory computer readable medium.

The memory 650 includes but is not limited to Read Only Memory (ROM), Random Access Memory (RAM), or a memory array including a combination of volatile and non-volatile memory units. The memory 650 may be utilized as working memory by the controller 610 while executing the processes and algorithms of the present disclosure. Additionally, the memory 650 may be used for long-term storage, e.g., of image data and information related thereto.

The device 600 includes a control line CL and data line DL as internal communication bus lines. Control data to/from the controller 610 may be transmitted through the control line CL. The data line DL may be used for transmission of voice data, displayed data, etc.

The antenna 601 transmits/receives electromagnetic wave signals between base stations for performing radio-based communication, such as the various forms of cellular telephone communication. The wireless communication processor 602 controls the communication performed between the device 600 and other external devices via the antenna 601. For example, the wireless communication processor 602 may control communication between base stations for cellular phone communication.

The speaker 604 emits an audio signal corresponding to audio data supplied from the voice processor 603. The microphone 605 detects surrounding audio and converts the detected audio into an audio signal. The audio signal may then be output to the voice processor 603 for further processing. The voice processor 603 demodulates and/or decodes the audio data read from the memory 650 or audio data received by the wireless communication processor 602 and/or a short-distance wireless communication processor 607. Additionally, the voice processor 603 may decode audio signals obtained by the microphone 605.

The exemplary device 600 may also include a display 620, a touch panel 630, an operation key 640, and a short-distance communication processor 607 connected to an antenna 606. The display 620 may be an LCD, an organic electroluminescence display panel, or another display screen technology. In addition to displaying still and moving image data, the display 620 may display operational inputs, such as numbers or icons which may be used for control of the device 600. The display 620 may additionally display a GUI for a user to control aspects of the device 600 and/or other devices. Further, the display 620 may display characters and images received by the device 600 and/or stored in the memory 650 or accessed from an external device on a network. For example, the device 600 may access a network such as the Internet and display text and/or images transmitted from a Web server.

The touch panel 630 may include a physical touch panel display screen and a touch panel driver. The touch panel 630 may include one or more touch sensors for detecting an input operation on an operation surface of the touch panel display screen. The touch panel 630 also detects a touch shape and a touch area. Used herein, the phrase “touch operation” refers to an input operation performed by touching an operation surface of the touch panel display with an instruction object, such as a finger, thumb, or stylus-type instrument. In the case where a stylus or the like is used in a touch operation, the stylus may include a conductive material at least at the tip of the stylus such that the sensors included in the touch panel 630 may detect when the stylus approaches/contacts the operation surface of the touch panel display (similar to the case in which a finger is used for the touch operation).

In certain aspects of the present disclosure, the touch panel 630 may be disposed adjacent to the display 620 (e.g., laminated) or may be formed integrally with the display 620. For simplicity, the present disclosure assumes the touch panel 630 is formed integrally with the display 620 and therefore, examples discussed herein may describe touch operations being performed on the surface of the display 620 rather than the touch panel 630. However, the skilled artisan will appreciate that this is not limiting.

For simplicity, the present disclosure assumes the touch panel 630 is a capacitance-type touch panel technology. However, it should be appreciated that aspects of the present disclosure may easily be applied to other touch panel types (e.g., resistance-type touch panels) with alternate structures. In certain aspects of the present disclosure, the touch panel 630 may include transparent electrode touch sensors arranged in the X-Y direction on the surface of transparent sensor glass.

The touch panel driver may be included in the touch panel 630 for control processing related to the touch panel 630, such as scanning control. For example, the touch panel driver may scan each sensor in an electrostatic capacitance transparent electrode pattern in the X-direction and Y-direction and detect the electrostatic capacitance value of each sensor to determine when a touch operation is performed. The touch panel driver may output a coordinate and corresponding electrostatic capacitance value for each sensor. The touch panel driver may also output a sensor identifier that may be mapped to a coordinate on the touch panel display screen. Additionally, the touch panel driver and touch panel sensors may detect when an instruction object, such as a finger is within a predetermined distance from an operation surface of the touch panel display screen. That is, the instruction object does not necessarily need to directly contact the operation surface of the touch panel display screen for touch sensors to detect the instruction object and perform processing described herein. For example, in an embodiment, the touch panel 630 may detect a position of a user's finger around an edge of the display panel 620 (e.g., gripping a protective case that surrounds the display/touch panel). Signals may be transmitted by the touch panel driver, e.g., in response to a detection of a touch operation, in response to a query from another element based on timed data exchange, etc.

The touch panel 630 and the display 620 may be surrounded by a protective casing, which may also enclose the other elements included in the device 600. In an embodiment, a position of the user's fingers on the protective casing (but not directly on the surface of the display 620) may be detected by the touch panel 630 sensors. Accordingly, the controller 610 may perform display control processing described herein based on the detected position of the user's fingers gripping the casing. For example, an element in an interface may be moved to a new location within the interface (e.g., closer to one or more of the fingers) based on the detected finger position.

Further, in an embodiment, the controller 610 may be configured to detect which hand is holding the device 600, based on the detected finger position. For example, the touch panel 630 sensors may detect a plurality of fingers on the left side of the device 600 (e.g., on an edge of the display 620 or on the protective casing), and detect a single finger on the right side of the device 600. In this exemplary scenario, the controller 610 may determine that the user is holding the device 600 with his/her right hand because the detected grip pattern corresponds to an expected pattern when the device 600 is held only with the right hand.

The operation key 640 may include one or more buttons or similar external control elements, which may generate an operation signal based on a detected input by the user. In addition to outputs from the touch panel 630, these operation signals may be supplied to the controller 610 for performing related processing and control. In certain aspects of the present disclosure, the processing and/or functions associated with external buttons and the like may be performed by the controller 610 in response to an input operation on the touch panel 630 display screen rather than the external button, key, etc. In this way, external buttons on the device 600 may be eliminated in lieu of performing inputs via touch operations, thereby improving watertightness.

The antenna 606 may transmit/receive electromagnetic wave signals to/from other external apparatuses, and the short-distance wireless communication processor 607 may control the wireless communication performed between the other external apparatuses. Bluetooth, IEEE 802.11, and near-field communication (NFC) are non-limiting examples of wireless communication protocols that may be used for inter-device communication via the short-distance wireless communication processor 607.

The device 600 may include a motion sensor 608. The motion sensor 608 may detect features of motion (i.e., one or more movements) of the device 600. For example, the motion sensor 608 may include an accelerometer to detect acceleration, a gyroscope to detect angular velocity, a geomagnetic sensor to detect direction, a geo-location sensor to detect location, etc., or a combination thereof to detect motion of the device 600. In an embodiment, the motion sensor 608 may generate a detection signal that includes data representing the detected motion. For example, the motion sensor 608 may determine a number of distinct movements in a motion (e.g., from start of the series of movements to the stop, within a predetermined time interval, etc.), a number of physical shocks on the device 600 (e.g., a jarring, hitting, etc., of the electronic device), a speed and/or acceleration of the motion (instantaneous and/or temporal), or other motion features. The detected motion features may be included in the generated detection signal. The detection signal may be transmitted, e.g., to the controller 610, whereby further processing may be performed based on data included in the detection signal. The motion sensor 608 can work in conjunction with a Global Positioning System (GPS) section 660. The information of the present position detected by the GPS section 660 is transmitted to the controller 610. An antenna 661 is connected to the GPS section 660 for receiving and transmitting signals to and from a GPS satellite.

The device 600 may include a camera section 609, which includes a lens and shutter for capturing photographs of the surroundings around the device 600. In an embodiment, the camera section 609 captures surroundings of an opposite side of the device 600 from the user. The images of the captured photographs can be displayed on the display panel 620. A memory section saves the captured photographs. The memory section may reside within the camera section 609 or it may be part of the memory 650. The camera section 609 can be a separate feature attached to the device 600 or it can be a built-in camera feature.

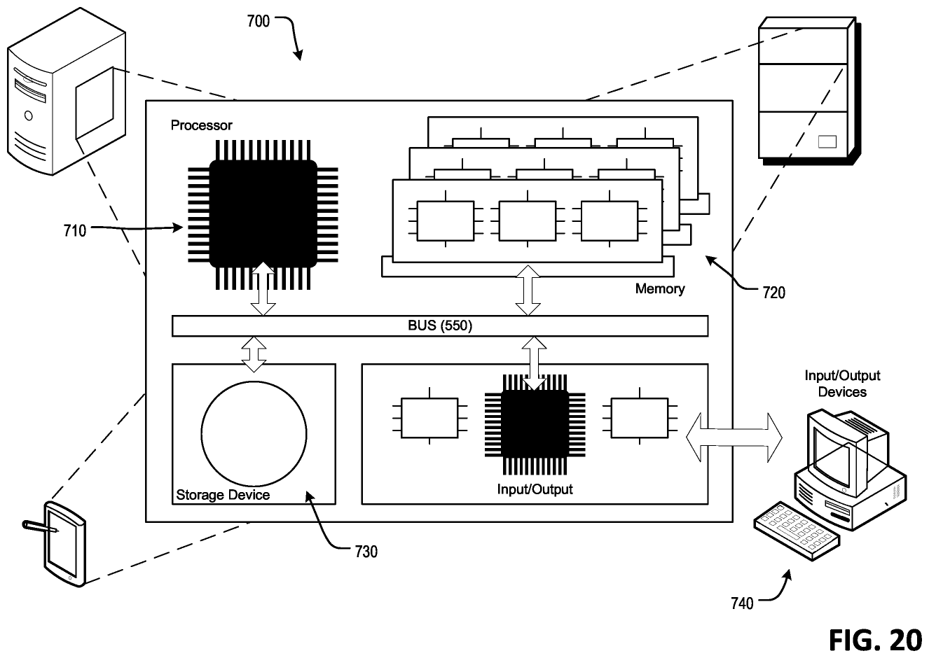

An example of a type of computer is shown in FIG. 20. The computer 700 can be used for the operations described in association with any of the computer-implement methods described previously, according to one implementation. For example, the computer 700 can be an example of devices 1805, 1815, or a server (such as device 1810). The computer 700 includes processing circuitry, as discussed above. The device 1815 may include other components not explicitly illustrated in FIG. 20 such as a CPU, GPU, main memory, frame buffer, etc. The processing circuitry includes one or more of the elements discussed next with reference to FIG. 20. In FIG. 20, the computer 700 includes a processor 710, a memory 720, a storage device 730, and an input/output device 740. Each of the components 710, 720, 730, and 740 are interconnected using a system bus 750. The processor 710 is capable of processing instructions for execution within the system 700. In one implementation, the processor 710 is a single-threaded processor. In another implementation, the processor 710 is a multi-threaded processor. The processor 710 is capable of processing instructions stored in the memory 720 or on the storage device 730 to display graphical information for a user interface on the input/output device 740.

The memory 720 stores information within the computer 700. In one implementation, the memory 720 is a computer-readable medium. In one implementation, the memory 720 is a volatile memory. In another implementation, the memory 720 is a non-volatile memory.

The storage device 730 is capable of providing mass storage for the system 700. In one implementation, the storage device 730 is a computer-readable medium. In various different implementations, the storage device 730 may be a floppy disk device, a hard disk device, an optical disk device, or a tape device.

The input/output device 740 provides input/output operations for the computer 700. In one implementation, the input/output device 740 includes a keyboard and/or pointing device. In another implementation, the input/output device 740 includes a display for displaying graphical user interfaces.

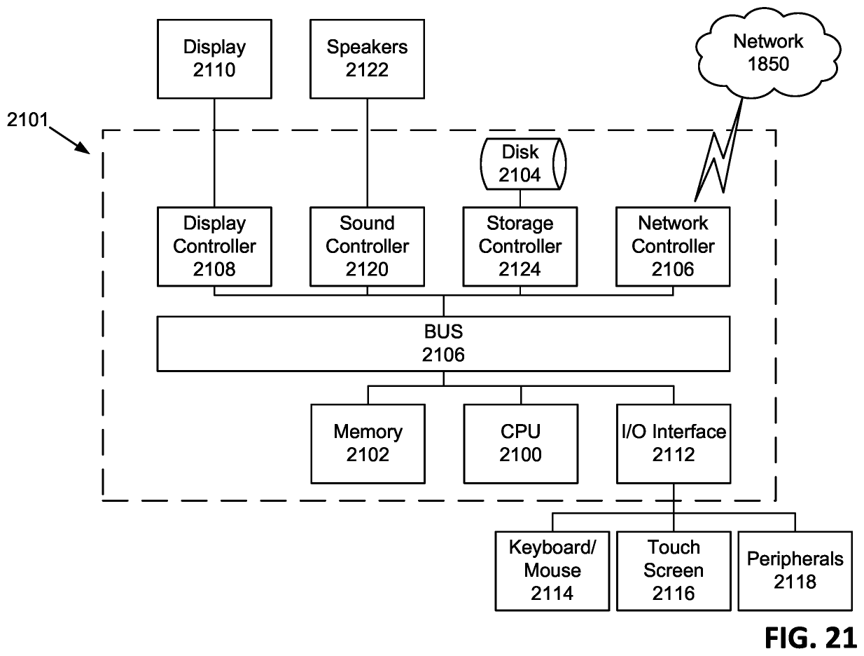

Next, a hardware description of a device 2101 according to exemplary embodiments is described with reference to FIG. 21. In FIG. 21, the device 2101, which can be the above described devices of FIG. 21, includes processing circuitry, as discussed above. The processing circuitry includes one or more of the elements discussed next with reference to FIG. 21. The device 2101 may include other components not explicitly illustrated in FIG. 21 such as a CPU, GPU, main memory, frame buffer, etc. In FIG. 21, the device 2101 includes a CPU 2100 which performs the processes described above/below. The process data and instructions may be stored in memory 2102. These processes and instructions may also be stored on a storage medium disk 2104 such as a hard drive (HDD) or portable storage medium or may be stored remotely. Further, the claimed advancements are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the device communicates, such as a server or computer.

Further, the claimed advancements may be provided as a utility application, background daemon, or component of an operating system, or combination thereof, executing in conjunction with CPU 2100 and an operating system such as Microsoft Windows, UNIX, Solaris, LINUX, Apple MAC-OS and other systems known to those skilled in the art.

The hardware elements in order to achieve the device may be realized by various circuitry elements, known to those skilled in the art. For example, CPU 2100 may be a Xenon or Core processor from Intel of America or an Opteron processor from AMD of America, or may be other processor types that would be recognized by one of ordinary skill in the art. Alternatively, the CPU 2100 may be implemented on an FPGA, ASIC, PLD or using discrete logic circuits, as one of ordinary skill in the art would recognize. Further, CPU 2100 may be implemented as multiple processors cooperatively working in parallel to perform the instructions of the processes described above.

The device 2101 in FIG. 12 also includes a network controller 2106, such as an Intel Ethernet PRO network interface card from Intel Corporation of America, for interfacing with the network 1850 (also shown in FIG. 18), and to communicate with the other devices of FIG. 18. As can be appreciated, the network 1850 can be a public network, such as the Internet, or a private network such as an LAN or WAN network, or any combination thereof and can also include PSTN or ISDN sub-networks. The network 1850 can also be wired, such as an Ethernet network, or can be wireless such as a cellular network including EDGE, 3G, 4G and 5G wireless cellular systems. The wireless network can also be WiFi, Bluetooth, or any other wireless form of communication that is known.

The device further includes a display controller 2108, such as a NVIDIA GeForce GTX or Quadro graphics adaptor from NVIDIA Corporation of America for interfacing with display 2110, such as an LCD monitor. A general purpose I/O interface 2112 interfaces with a keyboard and/or mouse 2114 as well as a touch screen panel 2116 on or separate from display 2110. General purpose I/O interface also connects to a variety of peripherals 2118 including printers and scanners.

A sound controller 2120 is also provided in the device to interface with speakers/microphone 2122 thereby providing sounds and/or music.

The general-purpose storage controller 2124 connects the storage medium disk 2104 with communication bus 2126, which may be an ISA, EISA, VESA, PCI, or similar, for interconnecting all of the components of the device. A description of the general features and functionality of the display 2110, keyboard and/or mouse 2114, as well as the display controller 2108, storage controller 2124, network controller 2106, sound controller 2120, and general purpose I/O interface 2112 is omitted herein for brevity as these features are known.

While this specification contains many specific implementation details, these should not be construed as limitations on the scope of what may be claimed, but rather as descriptions of features that may be specific to particular embodiments.

Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system modules and components in the embodiments described above should not be understood as requiring such separation in all embodiments, and it should be understood that the described program components and systems can generally be integrated together in a single component or packaged into multiple components.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

Obviously, numerous modifications and variations are possible in light of the above teachings. It is therefore to be understood that within the scope of the appended claims, embodiments of the present disclosure may be practiced otherwise than as specifically described herein.

Embodiments of the present disclosure may also be as set forth in the following parentheticals:

(1) A solution blow spin device, including a syringe pump assembly including: a parameter input interface configured to receive an input from a user, a first lead screw mechanically coupled to a first actuator and a second lead screw mechanically coupled to a second actuator, the first actuator and the second actuator each being electrically connected to the parameter input interface, the second lead screw being disposed proximal and parallel to the first lead screw, a first syringe coupled to a first syringe holder and a second syringe coupled to a second syringe holder, the first syringe holder and the second syringe holder disposed at a first end of the syringe pump assembly, a plunger of the first syringe extending out from the first syringe and a plunger of the second syringe extending out from the second syringe, the first syringe configured to hold a first solution and the second syringe configured to hold a second solution, the second syringe being disposed proximal and parallel to the second syringe, a first block coupled to the first lead screw, the first block configured to abut the plunger of the first syringe and translate along a direction of the first lead screw upon rotation of the first lead screw, and a second block coupled to the second lead screw, the second block configured to abut the plunger of the second syringe and translate along a direction of the second lead screw upon rotation of the second lead screw; and an airbrush adapter.

(2) The device of (1), wherein the airbrush adapter includes: an adapter base including a first portion, an open portion, and a second portion, the first portion of the adapter base coupling with the first end of the syringe pump assembly, a first syringe fitment configured to receive a tip of the first syringe, the first syringe fitment being an opening extending through the first portion, a first hollow needle fluidly connected to the first syringe, the first hollow needle disposed in a hollow nozzle body, the first hollow needle having a tip arranged through a first opening at a tip of the hollow nozzle body, the tip of the first hollow needle being concentric with the first opening but spaced apart from the first opening, the first hollow needle configured to eject the first solution through the tip of the first hollow needle, a second syringe fitment configured to receive a tip of the second syringe, the second syringe fitment being an opening extending through the first portion, a second hollow needle fluidly connected to the second syringe, the second hollow needle disposed in the hollow nozzle body, the second hollow needle having a tip arranged through a second opening at the tip of the hollow nozzle body, the tip of the second hollow needle being concentric with the second opening but spaced apart from the second opening, the second hollow needle configured to eject the second solution through the tip of the second hollow needle, and an air channel inlet fluidly coupled to the hollow nozzle body, the air channel inlet configured to receive a pressurized gas, the hollow nozzle body configured to eject the pressurized gas through the first opening around the tip of the first hollow needle and the second opening around the tip of the second hollow needle.

(3) The device of either (1) or (2), wherein the airbrush adapter includes a first channel adapter fluidly coupled to the first syringe fitment at an end of the first syringe fitment opposite the first syringe tip, and the airbrush adapter includes a second channel adapter fluidly coupled to the second syringe fitment at an end of the second syringe fitment opposite the second syringe tip.

(4) The device of any one of (1) to (3), wherein the airbrush adapter includes a first channel tube fluidly coupled to the first channel adapter and an end of the first hollow needle opposite the tip of the first hollow needle, and the airbrush adapter includes a second channel tube fluidly coupled to the second channel adapter and an end of the second hollow needle opposite the tip of the second hollow needle.

(5) The device of any one of (1) to (4), wherein the first syringe is fluidly coupled to the first hollow needle via the first syringe fitment, the first channel adapter, and the first channel tube, and the second syringe is fluidly coupled to the second hollow needle via the second syringe fitment, the second channel adapter, and the second channel tube.

(6) The device of any one of (1) to (5), wherein the parameter input interface is a display configured to display at least one button for receiving the input from the user.

(7) The device of any one of (1) to (6), wherein the parameter input interface is a surface of the syringe pump assembly including at least one physical button for receiving the input from the user.

(8) The device of any one of (1) to (7), wherein the first solution comprises a biocompatible polymer in a solvent.

(9) The device of any one of (1) to (8), wherein the biocompatible polymer is at least one selected from the group consisting of poly(lactic-co-glycolic acid), poly(ethylene glycol), and poly(lactide-co-caprolactone).

(10) The device of any one of (1) to (9), wherein the second solution comprises autologous skin cell suspensions.

(11) The device of any one of (1) to (10), wherein the first block is coupled to the first lead screw via an opening in the first block having internal threading and the second block is coupled to the second lead screw via an opening in the second block having internal threading.

(12) The device of any one of (1) to (11), wherein the syringe pump assembly includes a first rail disposed proximal and parallel to the first lead screw and a second rail disposed proximal and parallel to the second lead screw, the first block is additionally coupled to the first rail, the first rail configured to prevent a rotation of the first block upon rotation of the first lead screw, and the second block is additionally coupled to the second rail, the second rail configured to prevent a rotation of the second block upon rotation of the second lead screw.

(13) The device of any one of (1) to (12), wherein the first actuator is configured to rotate the first lead screw in a first rotation direction, the first block is configured to translate towards the first syringe and depress the plunger into the first syringe when the first lead screw rotates in the first rotation direction, and the first syringe is configured to eject the first solution through the first hollow needle when the plunger is depressed into the first syringe.

(14) The device of any one of (1) to (13), wherein a size of the first syringe holder is different from a size of the second syringe holder and a size of the first syringe is different from a size of the second syringe.

(15) The device of any one of (1) to (14), wherein the first syringe is a Luer syringe and a tip of the first syringe is a Luer slip tip.

(16) The device of any one of (1) to (15), wherein the syringe pump assembly includes a contact plate disposed at the first end of the syringe pump assembly, the contact plate configured to electrically couple with a complementary electrical contact.

(17) The device of any one of (1) to (16), further including a handle base, including a first portion configured to couple with the syringe pump assembly and a second portion configured to fluidly couple with the airbrush adapter, and a handle attached to the first portion of the handle base, the handle including a push button configured to actuate the first actuator and the second actuator when depressed.

(18) The device of any one of (1) to (17), wherein the handle base includes a pressure regulator fluidly coupled to the second portion, the pressure regulator configured to receive pressurized gas from an external gas source, regulate a pressure of the pressurized gas passing through the pressure regulator, and output pressure-regulated pressurized gas to the airbrush adapter via the air channel inlet.

(19) The device of any one of (1) to (18), wherein the handle base includes electrical pins configured to electrically couple with a contact plate on the syringe pump assembly, and the push button is configured to control both (i) the actuation of the first actuator and the second actuator, and (ii) the pressure regulator output.

(20) The device of any one of (1) to (19), wherein the handle base includes a male rail for slideably engaging with a female groove on the syringe pump assembly to couple the syringe pump assembly to the handle base, and the handle base includes a spring latch configured to secure the syringe pump assembly to the handle base and prevent sliding of the syringe pump assembly off of the handle base.

Thus, the foregoing discussion discloses and describes merely exemplary embodiments of the present disclosure. As will be understood by those skilled in the art, the present disclosure may be embodied in other specific forms without departing from the spirit thereof. Accordingly, the disclosure of the present disclosure is intended to be illustrative, but not limiting of the scope of the disclosure, as well as other claims. The disclosure, including any readily discernible variants of the teachings herein, defines, in part, the scope of the foregoing claim terminology such that no inventive subject matter is dedicated to the public.

1. A solution blow spin device, comprising:
a syringe pump assembly including:
a parameter input interface configured to receive an input from a user,
a first lead screw mechanically coupled to a first actuator and a second lead screw mechanically coupled to a second actuator, the first actuator and the second actuator each being electrically connected to the parameter input interface, the second lead screw being disposed proximal and parallel to the first lead screw,
a first syringe coupled to a first syringe holder and a second syringe coupled to a second syringe holder, the first syringe holder and the second syringe holder disposed at a first end of the syringe pump assembly, a plunger of the first syringe extending out from the first syringe and a plunger of the second syringe extending out from the second syringe, the first syringe configured to hold a first solution and the second syringe configured to hold a second solution, the second syringe being disposed proximal and parallel to the second syringe,
a first block coupled to the first lead screw, the first block configured to abut the plunger of the first syringe and translate along a direction of the first lead screw upon rotation of the first lead screw, and
a second block coupled to the second lead screw, the second block configured to abut the plunger of the second syringe and translate along a direction of the second lead screw upon rotation of the second lead screw; and
an airbrush adapter.

2. The device of claim 1, wherein the airbrush adapter includes:
an adapter base including a first portion, an open portion, and a second portion, the first portion of the adapter base coupling with the first end of the syringe pump assembly,
a first syringe fitment configured to receive a tip of the first syringe, the first syringe fitment being an opening extending through the first portion,
a first hollow needle fluidly connected to the first syringe, the first hollow needle disposed in a hollow nozzle body, the first hollow needle having a tip arranged through a first opening at a tip of the hollow nozzle body, the tip of the first hollow needle being concentric with the first opening but spaced apart from the first opening, the first hollow needle configured to eject the first solution through the tip of the first hollow needle,
a second syringe fitment configured to receive a tip of the second syringe, the second syringe fitment being an opening extending through the first portion,
a second hollow needle fluidly connected to the second syringe, the second hollow needle disposed in the hollow nozzle body, the second hollow needle having a tip arranged through a second opening at the tip of the hollow nozzle body, the tip of the second hollow needle being concentric with the second opening but spaced apart from the second opening, the second hollow needle configured to eject the second solution through the tip of the second hollow needle, and
an air channel inlet fluidly coupled to the hollow nozzle body, the air channel inlet configured to receive a pressurized gas, the hollow nozzle body configured to eject the pressurized gas through the first opening around the tip of the first hollow needle and the second opening around the tip of the second hollow needle.

3. The device of claim 2, wherein
the airbrush adapter includes a first channel adapter fluidly coupled to the first syringe fitment at an end of the first syringe fitment opposite the first syringe tip, and
the airbrush adapter includes a second channel adapter fluidly coupled to the second syringe fitment at an end of the second syringe fitment opposite the second syringe tip.

4. The device of claim 3, wherein
the airbrush adapter includes a first channel tube fluidly coupled to the first channel adapter and an end of the first hollow needle opposite the tip of the first hollow needle, and
the airbrush adapter includes a second channel tube fluidly coupled to the second channel adapter and an end of the second hollow needle opposite the tip of the second hollow needle.

5. The device of claim 4, wherein
the first syringe is fluidly coupled to the first hollow needle via the first syringe fitment, the first channel adapter, and the first channel tube, and
the second syringe is fluidly coupled to the second hollow needle via the second syringe fitment, the second channel adapter, and the second channel tube.

6. The device of claim 1, wherein the parameter input interface is a display configured to display at least one button for receiving the input from the user.

7. The device of claim 1, wherein the parameter input interface is a surface of the syringe pump assembly including at least one physical button for receiving the input from the user.

8. The device of claim 1, wherein the first solution comprises a biocompatible polymer in a solvent.

9. The device of claim 8, wherein the biocompatible polymer is at least one selected from the group consisting of poly(lactic-co-glycolic acid), poly(ethylene glycol), and poly(lactide-co-caprolactone).

10. The device of claim 8, wherein the second solution comprises autologous skin cell suspensions.

11. The device of claim 1, wherein the first block is coupled to the first lead screw via an opening in the first block having internal threading and the second block is coupled to the second lead screw via an opening in the second block having internal threading.

12. The device of claim 1, wherein
the syringe pump assembly includes a first rail disposed proximal and parallel to the first lead screw and a second rail disposed proximal and parallel to the second lead screw,
the first block is additionally coupled to the first rail, the first rail configured to prevent a rotation of the first block upon rotation of the first lead screw, and
the second block is additionally coupled to the second rail, the second rail configured to prevent a rotation of the second block upon rotation of the second lead screw.

13. The device of claim 2, wherein
the first actuator is configured to rotate the first lead screw in a first rotation direction,
the first block is configured to translate towards the first syringe and depress the plunger into the first syringe when the first lead screw rotates in the first rotation direction, and
the first syringe is configured to eject the first solution through the first hollow needle when the plunger is depressed into the first syringe.

14. The device of claim 1, wherein a size of the first syringe holder is different from a size of the second syringe holder and a size of the first syringe is different from a size of the second syringe.

15. The device of claim 1, wherein the first syringe is a Luer syringe and a tip of the first syringe is a Luer slip tip.

16. The device of claim 1, wherein the syringe pump assembly includes a contact plate disposed at the first end of the syringe pump assembly, the contact plate configured to electrically couple with a complementary electrical contact.

17. The device of claim 2, further comprising:
a handle base, including
a first portion configured to couple with the syringe pump assembly and a second portion configured to fluidly couple with the airbrush adapter, and
a handle attached to the first portion of the handle base, the handle including a push button configured to actuate the first actuator and the second actuator when depressed.

18. The device of claim 17, wherein the handle base includes a pressure regulator fluidly coupled to the second portion, the pressure regulator configured to receive pressurized gas from an external gas source, regulate a pressure of the pressurized gas passing through the pressure regulator, and output pressure-regulated pressurized gas to the airbrush adapter via the air channel inlet.

19. The device of claim 18, wherein
the handle base includes electrical pins configured to electrically couple with a contact plate on the syringe pump assembly, and
the push button is configured to control both (i) the actuation of the first actuator and the second actuator, and (ii) the pressure regulator output.

20. The device of claim 17, wherein
the handle base includes a male rail for slideably engaging with a female groove on the syringe pump assembly to couple the syringe pump assembly to the handle base, and
the handle base includes a spring latch configured to secure the syringe pump assembly to the handle base and prevent sliding of the syringe pump assembly off of the handle base.
